SEQUENCE LISTING (35 sequences)

SEQ ID NO: 1 - protein, 418 aa, Homo sapiens [sequence omitted]

SEQ ID NO: 2 - protein, 416 aa, Bos taurus
MQALVLLLWTGALLGFGRCQNAGQEAGSLTPESTGAPVEEEDPFFKVPVNKLAAAVSNFGYDLYRVRSGESPTANVLLSPLSVATALSALSLGAEQRTESNIHRALYYDLISNPDIHGTYKDLLASVTAPQKNLKSASRIIFERKLRIKASFIPPLEKSYGTRPRILTGNSRVDLQEINNWVQAQMKGKVARSTREMPSEISIFLLGVAYFKGQWVTKFDSRKTSLEDFYLDEERTVKVPMMSDPQAVLRYGLDSDLNCKIAQLPLTGSTSIIFFLPQKVTQNLTLIEESLTSEFIHDIDRELKTVQAVLTIPKLKLSYEGELTKSVQELKLQSLFDAPDFSKITGKPIKLTQVEHRVGFEWNEDGAGTNSSPGVQPARLTFPLDYHLNQPFIFVLRDTDTGALLFIGKILDPRGT

SEQ ID NO: 3 - protein, 417 aa, Mus musculus
MQALVLLLWTGALLGHGSSQNVPSSSEGSPVPDSTGEPVEEEDPFFKVPVNKLAAAVSNFGYDLYRLRSSASPTGNVLLSPLSVATALSALSLGAEHRTESVIHRALYYDLITNPDIHSTYKELLASVTAPEKNLKSASRIVFERKLRVKSSFVAPLEKSYGTRPRILTGNPRVDLQEINNWVQAQMKGKIARSTREMPSALSILLLGVAYFKGQWVTKFDSRKTTLQDFHLDEDRTVRVPMMSDPKAILRYGLDSDLNCKIAQLPLTGSMSIIFFLPLAVTQNLTMIEESLTSEFIHDIDRELKTIQAVLTVPKLKLSFEGELTKSLQDMKLQSLFESPDFSKITGKPVKLTQVEHRAAFEWNEEGAGSSPSPGLQPVRLTFPLDYHLNQPFLFVLRDTDTGALLFIGRILDPSST

SEQ ID NO: 4 - protein, 480 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEED

SEQ ID NO: 5 - protein, 184 aa, Homo sapiens
MHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFNGKDVLTHPTWPQKSVWHGSDPNGRRLTESYCETWRTEAPSATGQAYSLLGGRLLGQSAASCHHAYIVLCIENSFMTASK

SEQ ID NO: 6 - protein, 184 aa, Mus musculus
HTHQDFQPVLHLVALNTPLSGGMRGIRGADFQCFQQARAVGLSGTFRAFLSSRLQDLYSIVRRADRGSVPIVNLKDEVLSPSWDSLFSGSQGQLQPGARIFSFDGRDVLRHPAWPQKSVWHGSDPSGRRLMESYCETWRTETTGATGQASSLLSGRLLEQKAASCHNSYIVLCIENSFMTSFSK

SEQ ID NO: 7 - protein, 211 aa, Bos taurus
MTPWLGLVVLLGSWSLGDWGAEACTCSPSHPQDAFCNSDIVIRAKVVGKKLLKEGPFGTMVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMFSNFGYPGYQSKHYACIRQKGGYCSWYRGWAPPDKSIINATDP

SEQ ID NO: 8 - protein, 211 aa, Rattus norvegicus
MTPWLGLVVLLSCWSLGHWGTEACTCSPSHPQDAFCNSDIVIRAKVVGKKLVKEGPFGTLVYTIKQMKMYRGFSKMPHVQYIHTEASESLCGLKLEVNKYQYLLTGRVYEGKMYTGLCNFVERWDHLTLSQRKGLNYRYHLGCNCKIKSCYYLPCFVTSKKECLWTDMLSNFGYPGYQSKHYACIRQKGGYCSWYRGWAPPDKSISNATDP

SEQ ID NO: 9 - protein, 687 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRGEHCNKKAVFSRISKFKSTRNDCTTQSNVKH

SEQ ID NO: 10 - protein, 688 aa, Mus musculus
MVSCWDTAVLPYALLGCLLLTGYGSGSKLKVPELSLKGTQHVMQAGQTLFLKCRGEAAHSWSLPTTVSQEDKRLSITPPSACGRDNRQFCSTLTLDTAQANHTGLYTCRYLPTSTSKKKKAESSIYIFVSDAGSPFIEMHTDIPKLVHMTEGRQLIIPCRVTSPNVTVTLKKFPFDTLTPDGQRITWDSRRGFIIANATYKEIGLLNCEATVNGHLYQTNYLTHRQTNTILDVQIRPPSPVRLLHGQTLVLNCTATTELNTRVQMSWNYPGKATKRASIRQRIDRSHSHNNVFHSVLKINNVESRDKGLYTCRVKSGSSFQSFNTSVHVYEKGFISVKHRKQPVQETTAGRRSYRLSMKVKAFPSPEIVWLKDGSPATLKSARYLVHGYSLIIKDVTTEDAGDYTILLGIKQSRLFKNLTATLIVNVKPQIYEKSVSSLPSPPLYPLGSRQVLTCTVYGIPRPTITWLWHPCHHNHSKERYDFCTENEESFILDPSSNLGNRIESISQRMTVIEGTNKTVSTLVVADSQTPGIYSCRAFNKIGTVERNIKFYVTDVPNGFHVSLEKMPAEGEDLKLSCVVNKFLYRDITWILLRTVNNRTMHHSISKQKMATTQDYSITLNLVIKNVSLEDSGTYACRARNIYTGEDILRKTEVLVRGEHCGKKAIFSRISKFKSRRNDCTTQSHVKH

SEQ ID NO: 11 - protein, 12 aa, Homo sapiens
KGKGQKRKRKKS

SEQ ID NO: 12 - protein, 24 aa, Homo sapiens
CSCKNTDSRCKARQLELNERTCRC

SEQ ID NO: 13 - protein, 931 aa, Homo sapiens
MRKGLRATAARCGLGLGYLLQMLVLPALALLSASGTGSAAQDDDFFHELPETFPSDPPEPLPHFLIEPEEAYIVKNKPVNLYCKASPATQIYFKCNSEWVHQKDHIVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPVEDRNFYITIDHNLIIKQARLSDTANYTCVAKNIVAKRKSTTATVIVYVNGGWSTWTEWSVCNSRCGRGYQKRTRTCTNPAPLNGGAFCEGQSVQKIACTTLCPVDGRWTPWSKWSTCGTECTHWRRRECTAPAPKNGGKDCDGLVLQSKNCTDGLCMQTAPDSDDVALYVGIVIAVIVCLAISVVVALFVYRKNHRDFESDIIDSSALNGGFQPVNIKAARQDLLAVPPDLTSAAAMYRGPVYALHDVSDKIPMTNSPILDPLPNLKIKVYNTSGAVTPQDDLSEFTSKLSPQMTQSLLENEALSLKNQSLARQTDPSCTAFGSFNSLGGHLIVPNSGVSLLIPAGAIPQGRVYEMYVTVHRKETMRPPMDDSQTLLTPVVSCGPPGALLTRPVVLTMHHCADPNTEDWKILLKNQAAQGQWEDVVVVGEENFTTPCYIKLDAEACHILTENLSTYALVGHSTTKAAAKRLKLAIFGPLCCSSLEYSIRVYCLDDTQDALKEILHLERQTGGQLLEEPKALHFKGSTHNLRLSIHDIAHSLWKSKLLAKYQEIPFYHVWSGSQRNLHCTFTLERFSLNTVELVCKLCVRQVEGEGQIFQLNCTVSEEPTGIDLPLLDPANTITTVTGPSAFSIPLPIRQKLCSSLDAPQTRGHDWRMLAHKLNLDRYLNYFATKSSPTGVILDLWEAQNFPDGNLSMLAAVLEEMGRHETVVSLAAEGQY

SEQ ID NO: 14 - protein, 471 aa, Homo sapiens
MPNSEPASLLELFNSIATQGELVRSLKAGNASKDEIDSAVKMLVSLKMSYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYGDAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 15 - protein, 528 aa, Homo sapiens
MGDAPSPEEKLHLITRNLQEVLGEEKLKEILKERELKIYWGTATTGKPHVAYFVPMSKIADFLKAGCEVTILFADLHAYLDNMKAPWELLELRVSYYENVIKAMLESIGVPLEKLKFIKGTDYQLSKEYTLDVYRLSSVVTQHDSKKAGAEVVKQVEHPLLSGLLYPGLQALDEEYLKVDAQFGGIDQRKIFTFAEKYLPALGYSKRVHLMNPMVPGLTGSKMSSSEEESKIDLLDRKEDVKKKLKKAFCEPGNVENNGVLSFIKHVLFPLKSEFVILRDEKWGGNKTYTAYVDLEKDFAAEVVHPGDLKNSVEVALNKLLDPIREKFNTPALKKLASAAYPDPSKQKPMAKGPAKNSEPEEVIPSRLDIRVGKIITVEKHPDADSLYVEKIDVGEAEPRTVVSGLVQFVPKEELQDRLVVVLCNLKPQKMRGVESQGMLLCASIEGINRQVEPLDPPAGSAPGEHVFVKGYEKGQPDEELKPKKKVFEKLQADFKISEECIAQWKQTNFMTKLGSISCKSLKGGNIS

SEQ ID NO: 16 - protein, 644 aa, Homo sapiens
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSGIK

SEQ ID NO: 17 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 18 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 19 - DNA, 1257 bp, Homo sapiens
atgcaggccctggtgctactcctctgcattggagccctcctcgggcacagcagctgccagaaccctgccagccccccggaggagggctccccagaccccgacagcacaggggcgctggtggaggaggaggatcctttcttcaaagtccccgtgaacaagctggcagcggctgtctccaacttcggctatgacctgtaccgggtgcgatccagcatgagccccacgaccaacgtgctcctgtctcctctcagtgtggccacggccctctcggccctctcgctgggagcggagcagcgaacagaatccatcattcaccgggctctctactatgacttgatcagcagcccagacatccatggtacctataaggagctccttgacacggtcactgccccccagaagaacctcaagagtgcctcccggatcgtctttgagaagaagctrcgcataaaatccagctttgtggcacctctggaaaagtcatatgggaccaggcccagagtcctgacgggcaaccctcgcttggacctgcaagagatcaacaactgggtgcaggcgcagatgaaagggaagctcgccaggtccacaaaggaaattcccgatgagatcagcattctccttctcggtgtggcgcacttcaaggggcagtgggtaacaaagtttgactccagaaagacttccctcgaggatttctacttggatgaagagaggaccgtgagggtccccatgatgtcggaccctaaggctgttttacgctatggcttggattcagatctcagctgcaagattgcccagctgcccttgaccggaagcatgagtatcatcttcttcctgcccctgaaagtgacccagaatttgaccttgatagaggagagcctcacctccgagttcattcatgacatagaccgagaactgaagaccgtgcaggcggtcctcactgtccccaagctgaagctgagttacgaaggcgaagtcaccaagtccctgcaggagatgaagctgcaatccttgtttgattcaccagactttagcaagatcacaggcaaacccatcaagctgactcaggtggaacaccgggctggctttgagtggaacgaggatggggcgggaaccacccccagcccagggctgcagcctgcccacctcaccttcccgctggactatcaccttaaccagcctttcatcttcgtactgagggacacagacacaggggcccttctcttcattggcaagattctggaccccaggggcccctaa

SEQ ID NO: 20 - DNA, 1251 bp, Bos taurus
atgcaggccctcgtgctactcctctggactggagccctgcttgggtttggccgctgccagaacgccggccaggaggcgggctctctgacccctgagagcacgggggcaccagtggaggaagaggatcccttcttcaaggtccctgtgaacaagctggcggcagcggtctccaacttcggctacgacctgtaccgcgtgagatccggtgagagccccaccgccaatgtgctgctgtctccgctcagcgtggccacggcgctctctgccctgtcgctgggtgcggaacagcggacagaatccaacattcaccgggctctgtactacgacctgatcagtaacccagacatccacggcacctacaaggacctccttgcctccgtcaccgccccccagaagaaccttaagagtgcttcccggattatctttgagaggaagctgcggataaaagccagcttcatcccacccctggagaagtcatatgggaccaggcccagaatcctgaccggcaactctcgagtagaccttcaggagattaacaactgggtgcaggcccagatgaaagggaaagtcgctaggtccacgagggagatgcccagtgagatcagcattttcctcctgggcgtggcttacttcaaggggcagtgggtaacaaagtttgactccagaaaaacttccctggaggatttctacttggatgaggagaggaccgtgaaagtccccatgatgtcagaccctcaggccgttttacggtacggcttggattctgatctcaactgcaagatcgcccagctgcccttgaccgggagcacaagtatcatcttcttcctgcctcagaaagtgacccagaacttgaccttgatagaagagagcctcacctctgagttcattcatgacatagaccgagaactgaagactgttcaggcggtcctgaccattcccaagctgaagctgagttatgaaggcgaactcacgaagtccgtgcaggagctgaagctgcaatccctgtttgatgcaccagactttagcaagatcacaggcaaacctatcaaacttactcaagtggaacatcgcgtcggatttgagtggaatgaggatggggcgggtactaactccagcccaggggtccagcctgcccgcctcaccttccctctggactatcaccttaaccaacctttcatctttgtactgagggacacagacacaggggcccttctcttcataggcaaaattctggaccccaggggcacttag

SEQ ID NO: 21 - DNA, 1254 bp, Mus musculus
atgcaggccctggtgctactcctctggactggagccctgctcgggcacggcagcagccagaacgtccccagcagctctgagggctccccagtcccggacagcacgggcgagcccgtggaggaggaggaccccttcttcaaggtccctgtgaacaagctggcagcagctgtctccaacttcggctacgatctgtaccgcctgagatccagtgccagcccaacgggcaacgtcctgctgtctccactcagcgtggccacggccctctctgccctttctctgggagctgaacatcgaacagagtctgtcattcaccgggctctctactacgacctgatcaccaaccctgacatccacagcacctacaaggagctccttgcctctgttactgcccctgagaagaacctcaagagtgcttccagaattgtgtttgagaggaaacttcgagtcaaatccagctttgttgcccctctggagaagtcctatgggaccaggccccggatcctcacgggcaaccctcgagtagaccttcaggagattaacaactgggtgcaggcccagatgaaagggaagattgcccggtccacgagggaaatgcccagtgccctcagcatccttctccttggcgtggcttacttcaaggggcagtgggtaaccaagtttgactcgagaaagacgaccctccaggattttcatttggacgaggacaggaccgtgagagtccccatgatgtcagatcctaaggccatcttacgatacggcttggactctgatctcaactgcaagattgcccagctgcccttgacaggaagtatgagcatcatcttcttcctgcccctgaccgtgacccagaacttgaccatgatagaagagagcctcacctctgagttcattcatgacatcgaccgagaactgaagactatccaagctgtgctgactgtccccaagctgaagctgagcttcgaaggcgaacttaccaagtctctgcaggacatgaagctacagtcgttgtttgaatcacccgacttcagcaagattactggcaaacccgtgaagctcacccaagtggaacacagggctgctttcgagtggaatgaagagggggcaggaagcagccccagcccaggcctccagcccgtccgcctcaccttcccgctagactatcaccttaaccaacctttcctctttgttctgagggacacggacacgggggccctcctcttcataggcagaatcctggaccccagtagcacttaa

SEQ ID NO: 22 - DNA, 555 bp, Homo sapiens
atgcacagccaccgcgacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttaacggcaaggacgtcctgacccaccccacctggccccagaagagcgtgtggcatggctcggaccccaacgggcgcaggctgaccgagagctactgtgagacgtggcggacggaggctccctcggccacgggccaggcctactcgctgctggggggcaggctcctggggcagagtgccgcgagctgccatcacgcctacatcgtgctatgcattgagaacagcttcatgactgcctccaagtag

SEQ ID NO: 23 - DNA, 552 bp, Mus musculus
catactcatcaggactttcagccagtgctccacctggtggcactgaacacccccctgtctggaggcatgcgtggtatccgtggagcagatttccagtgcttccagcaagcccgagccgtggggctgtcgggcaccttccgggctttcctgtcctctaggctgcaggatctctatagcatcgtgcgccgtgctgaccgggggtctgtgcccatcgtcaacctgaaggacgaggtgctatctcccagctgggactccctgttttctggctcccagggtcaactgcaacccggggcccgcatcttttcttttgacggcagagatgtcctgagacacccagcctggccgcagaagagcgtatggcacggctcggaccccagtgggcggaggctgatggagagttactgtgagacatggcgaactgaaactactggggctacaggtcaggcctcctccctgctgtcaggcaggctcctggaacagaaagctgcgagctgccacaacagctacatcgtcctgtgcattgagaatagcttcatgacctctttctccaaa

SEQ ID NO: 24 - DNA, 567 bp, Bos taurus
tgcacatgctcgcctagccacccccaggacgcgttctgcaactcagacatcgtgatccgagccaaggtggtagggaagaaactgctgaaggaggggccctttggcacgatggtctacaccatcaagcagatgaagatgtaccgaggattcaccaagatgccccatgtgcagtacatccacacagaagcttctgaaagtctctgtggccttaagcttgaggtcaacaagtaccagtacctgctgacaggccgagtctatgatggcaagatgtacacaggactgtgtaactttgtagagaggtgggaccagctcaccctctcccagcgcaaggggctgaactatcgatatcacctgggctgtaactgcaagatcaaatcctgctactacctgccttgctttgtaacctccaagaacgagtgtctctggaccgacatgttctccaatttcggctaccctggctaccagtccaaacactacgcttgcatccggcagaagggtggctactgtagctggtaccgaggatgggcacccccggacaaaagcatcatcaatgccacagacccctga

SEQ ID NO: 25 - DNA, 636 bp, Rattus norvegicus
atgactccctggcttgggcttgtcgtgctcctgagctgctggagccttgggcactggggaacggaagcgtgcacatgctcgcccagccatccccaggatgccttctgcaactccgacatcgtgatccgggccaaagtggtgggaaagaagctggtgaaggaagggccctttggcactctggtctacactattaagcaaatgaagatgtaccgaggattcagtaagatgccccatgtgcagtacattcacacggaagcctctgaaagtctctgtggccttaagctagaagtcaacaaataccagtacctgctgacagggcgcgtgtatgaaggcaagatgtacacagggctgtgcaactttgtggagaggtgggaccacctcacactgtcccagcgcaagggcctcaattaccgctaccacctgggttgcaattgcaagatcaagtcctgctactacttgccttgctttgtgacctccaagaaagaatgtctctggaccgacatgctctccaatttcgggtaccctggctatcagtccaaacactacgcctgcatccggcagaagggtggctactgcagctggtaccgaggatgggcccccccagacaagagcatcagcaatgccacagacccctga

SEQ ID NO: 26 - DNA, 2064 bp, Homo sapiens
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagaggtgagcactgcaacaaaaaggctgttttctctcggatctccaaatttaaaagcacaaggaatgattgtaccacacaaagtaatgtaaaacattaa

SEQ ID NO: 27 - DNA, 2067 bp, Mus musculus
atggtcagctgctgggacaccgcggtcttgccttacgcgctgctcgggtgtctgcttctcacaggatatggctcagggtcgaagttaaaagtgcctgaactgagtttaaaaggcacccagcatgtcatgcaagcaggccagactctctttctcaagtgcagaggggaggcagcccactcatggtctctgcccacgaccgtgagccaggaggacaaaaggctgagcatcactcccccatcggcctgtgggagggataacaggcaattctgcagcaccttgaccttggacacggcgcaggccaaccacacgggcctctacacctgtagatacctccctacatctacttcgaagaaaaagaaagcggaatcttcaatctacatatttgttagtgatgcagggagtcctttcatagagatgcacactgacatacccaaacttgtgcacatgacggaaggaagacagctcatcatcccctgccgggtgacgtcacccaacgtcacagtcaccctaaaaaagtttccatttgatactcttacccctgatgggcaaagaataacatgggacagtaggagaggctttataatagcaaatgcaacgtacaaagagataggactgctgaactgcgaagccaccgtcaacgggcacctgtaccagacaaactatctgacccatcggcagaccaatacaatcctagatgtccaaatacgcccgccgagcccagtgagactgctccacgggcagactcttgtcctcaactgcaccgccaccacggagctcaatacgagggtgcaaatgagctggaattaccctggtaaagcaactaagagagcatctataaggcagcggattgaccggagccattcccacaacaatgtgttccacagtgttcttaagatcaacaatgtggagagccgagacaaggggctctacacctgtcgcgtgaagagtgggtcctcgttccagtctttcaacacctccgtgcatgtgtatgaaaaaggattcatcagtgtgaaacatcggaagcagccggtgcaggaaaccacagcaggaagacggtcctatcggctgtccatgaaagtgaaggccttcccctccccagaaatcgtatggttaaaagatggctcgcctgcaacattgaagtctgctcgctatttggtacatggctactcattaattatcaaagatgtgacaaccgaggatgcaggggactatacgatcttgctgggcataaagcagtcaaggctatttaaaaacctcactgccactctcattgtaaacgtgaaacctcagatctacgaaaagtccgtgtcctcgcttccaagcccacctctctatccgctgggcagcagacaagtcctcacttgcaccgtgtatggcatccctcggccaacaatcacgtggctctggcacccctgtcaccacaatcactccaaagaaaggtatgacttctgcactgagaatgaagaatcctttatcctggatcccagcagcaacttaggaaacagaattgagagcatctctcagcgcatgacggtcatagaaggaacaaataagacggttagcacattggtggtggctgactctcagacccctggaatctacagctgccgggccttcaataaaatagggactgtggaaagaaacataaaattttacgtcacagatgtgccgaatggctttcacgtttccttggaaaagatgccagccgaaggagaggacctgaaactgtcctgtgtggtcaataaattcctgtacagagacattacctggattctgctacggacagttaacaacagaaccatgcaccatagtatcagcaagcaaaaaatggccaccactcaagattactccatcactctgaaccttgtcatcaagaacgtgtctctagaagactcgggcacctatgcgtgcagagccaggaacatatacacaggggaagacatccttcggaagacagaagttctcgttagaggtgagcactgcggcaaaaaggccattttctctcggatctccaaatttaaaagcaggaggaatgattgtaccacacaaagtcatgtcaaacattaa

SEQ ID NO: 28 - DNA, 36 bp, Homo sapiens
aagggaaaggggcaaaaacgaaagcgcaagaaatcc

SEQ ID NO: 29 - DNA, 72 bp, Homo sapiens
tgttcctgcaaaaacacagactcgcgttgcaaggcgaggcagcttgagttaaacgaacgtacttgcagatgt

SEQ ID NO: 30 - DNA, 2796 bp, Homo sapiens
atgaggaaaggtctgcgggcgacagcggcccgctgcggactgggactgggatacttgctgcaaatgctcgtgctacctgccctggccctgctcagcgccagcggcactggctccgccgcccaagatgatgacttttttcatgaactcccagaaacttttccttctgatccacctgagcctctgccacatttccttattgagcctgaagaagcttatattgtgaagaataagcctgtgaacctgtactgtaaagcaagccctgccacccagatctatttcaagtgtaatagtgaatgggttcatcagaaggaccacatagtagatgaaagagtagatgaaacttccggtctcattgtccgggaagtgagcattgagatttcgcgccagcaagtggaagaactctttggacctgaagattactggtgccagtgtgtggcctggagctccgcgggtaccacaaagagccggaaggcgtatgtgcgcattgcatatctacggaagacatttgagcaggaacccctaggaaaggaagtgtctttggaacaggaagtcttactccagtgtcgaccacctgaagggatcccagtggctgaggtggaatggttgaaaaatgaagacataattgatcccgttgaagatcggaatttttatattactattgatcacaacctcatcataaagcaggcccgactctctgatactgcaaattacacctgtgttgccaaaaacattgttgccaagaggaaaagtacaactgccactgtcatagtctatgtcaacggtggctggtccacctggacggagtggtctgtgtgtaacagccgctgtggacgagggtatcagaaacgtacaaggacttgtaccaacccggcaccactcaatgggggtgccttctgtgaagggcagagtgtgcagaaaatagcctgtactacgttatgcccagtggatggcaggtggacgccatggagcaagtggtctacttgtggaactgagtgcacccactggcgcaggagggagtgcacggcgccagcccccaagaatggaggcaaggactgcgacggcctcgtcttgcaatccaagaactgcactgatgggctttgcatgcagactgctcctgattcagatgatgttgctctctatgttgggattgtgatagcagtgatcgtttgcctggcgatctctgtagttgtggccttgtttgtgtatcggaagaatcatcgtgactttgagtcagatattattgactcttcggcactcaatgggggctttcagcctgtgaacatcaaggcagcaagacaagatctgctggctgtacccccagacctcacgtcagctgcagccatgtacagaggacctgtctatgccctgcatgacgtctcagacaaaatcccaatgaccaactctccaattctggatccactgcccaacctgaaaatcaaagtgtacaacacctcaggtgctgtcaccccccaagatgacctctctgagtttacgtccaagctgtcccctcagatgacccagtcgttgttggagaatgaagccctcagcctgaagaaccagagtctagcaaggcagactgatccatcctgtaccgcatttggcagcttcaactcgctgggaggtcaccttattgttcccaattcaggagtcagcttgctgattcccgctggggccattccccaagggagagtctacgaaatgtatgtgactgtacacaggaaagaaactatgaggccacccatggatgactctcagacacttttgacccctgtggtgagctgtgggcccccaggagctctgctcacccgccccgtcgtcctcactatgcatcactgcgcagaccccaataccgaggactggaaaatactgctcaagaaccaggcagcacagggacagtgggaggatgtggtggtggtcggggaggaaaacttcaccaccccctgctacattaagctggatgcagaggcctgccacatcctcacagagaacctcagcacctacgccctggtaggacattccaccaccaaagcggctgcaaagcgcctcaagctggccatctttgggcccctgtgctgctcctcgctggagtacagcatccgagtctactgtctggatgacacccaggatgccctgaaggaaattttacatcttgagagacagacgggaggacagctcctagaagaacctaaggctcttcattttaaaggcagcacccacaacctgcgcctgtcaattcacgatatcgcccattccctctggaagagcaaattgctggctaaatatcaggaaattccattttaccatgtttggagtggatctcaaagaaacctgcactgcaccttcactctggaaagatttagcctgaacacagtggagctggtttgcaaactctgtgtgcggcaggtggaaggagaagggcagatcttccagctcaactgcaccgtgtcagaggaacctactggcatcgatttgccgctgctggatcctgcgaacaccatcaccacggtcacggggcccagtgctttcagcatccctctccctatccggcagaagctctgtagcagcctggatgccccccagacgagaggccatgactggaggatgctggcccataagctgaacctggacaggtacttgaattactttgccaccaaatccagcccaactggcgtaatcctggatctttgggaagcacagaacttcccagatggaaacctgagcatgctggcagctgtcttggaagaaatgggaagacatgaaacggtggtgtccttagcagcagaagggcagtattaa

SEQ ID NO: 31 - DNA, 1416 bp, Homo sapiens
atgcccaacagtgagcccgcatctctgctggagctgttcaacagcatcgccacacaaggggagctcgtaaggtccctcaaagcgggaaatgcgtcaaaggatgaaattgattctgcagtaaagatgttggtgtcattaaaaatgagctacaaagctgccgcgggggaggattacaaggctgactgtcctccagggaacccagcacctaccagtaatcatggcccagatgccacagaagctgaagaggattttgtggacccatggacagtacagacaagcagtgcaaaaggcatagactacgataagctcattgttcggtttggaagtagtaaaattgacaaagagctaataaaccgaatagagagagccaccggccaaagaccacaccacttcctgcgcagaggcatcttcttctcacacagagatatgaatcaggttcttgatgcctatgaaaataagaagccattttatctgtacacgggccggggcccctcttctgaagcaatgcatgtaggtcacctcattccatttattttcacaaagtggctccaggatgtatttaacgtgcccttggtcatccagatgacggatgacgagaagtatctgtggaaggacctgaccctggaccaggcctatggcgatgctgttgagaatgccaaggacatcatcgcctgtggctttgacatcaacaagactttcatattctctgacctggactacatggggatgagctcaggtttctacaaaaatgtggtgaagattcaaaagcatgttaccttcaaccaagtgaaaggcattttcggcttcactgacagcgactgcattgggaagatcagttttcctgccatccaggctgctccctccttcagcaactcattcccacagatcttccgagacaggacggatatccagtgccttatcccatgtgccattgaccaggatccttactttagaatgacaagggacgtcgcccccaggatcggctatcctaaaccagccctgttgcactccaccttcttcccagccctgcagggcgcccagaccaaaatgagtgccagcgacccaaactcctccatcttcctcaccgacacggccaagcagatcaaaaccaaggtcaataagcatgcgttttctggagggagagacaccatcgaggagcacaggcagtttgggggcaactgtgatgtggacgtgtctttcatgtacctgaccttcttcctcgaggacgacgacaagctcgagcagatcaggaaggattacaccagcggagccatgctcaccggtgagctcaagaaggcactcatagaggttctgcagcccttgatcgcagagcaccaggcccggcgcaaggaggtcacggatgagatagtgaaagagttcatgactccccggaagctgtccttcgactttcagtag

SEQ ID NO: 32 - DNA, 1587 bp, Homo sapiens
atgggggacgctcccagccctgaagagaaactgcaccttatcacccggaacctgcaggaggttctgggggaagagaagctgaaggagatactgaaggagcgggaacttaaaatttactggggaacggcaaccacgggcaaaccacatgtggcttactttgtgcccatgtcaaagattgcagacttcttaaaggcagggtgtgaggtaacaattctgtttgcggacctccacgcatacctggataacatgaaagccccatgggaacttctagaactccgagtcagttactatgagaatgtgatcaaagcaatgctggagagcattggtgtgcccttggagaagctcaagttcatcaaaggcactgattaccagctcagcaaagagtacacactagatgtgtacagactctcctccgtggtcacacagcacgattccaagaaggctggagctgaggtggtaaagcaggtggagcaccctttgctgagtggcctcttataccccggactgcaggctttggatgaagagtatttaaaagtagatgcccaatttggaggcattgatcagagaaagattttcacctttgcagagaagtacctccctgcacttggctattcaaaacgggtccatctgatgaatcctatggttccaggattaacaggcagcaaaatgagctcttcagaagaggagtccaagattgatctccttgatcggaaggaggatgtgaagaaaaaactgaagaaggccttctgtgagccaggaaatgtggagaacaatggggttctgtccttcatcaagcatgtcctttttccccttaagtccgagtttgtgatcctacgagatgagaaatggggtggaaacaaaacctacacagcttacgtggacctggaaaaggactttgctgctgaggttgtacatcctggagacctgaagaattctgttgaagtcgcactgaacaagttgctggatccaatccgggaaaagtttaatacccctgccctgaaaaaactggccagcgctgcctacccagatccctcaaagcagaagccaatggccaaaggccctgccaagaattcagaaccagaggaggtcatcccatcccggctggatatccgtgtggggaaaatcatcactgtggagaagcacccagatgcagacagcctgtatgtagagaagattgacgtgggggaagctgaaccacggactgtggtgagcggcctggtacagttcgtgcccaaggaggaactgcaggacaggctggtagtggtgctgtgcaacctgaaaccccagaagatgagaggagtcgagtcccaaggcatgcttctgtgtgcttctatagaagggataaaccgccaggttgaacctctggaccctccggcaggctctgctcctggtgagcacgtgtttgtgaagggctatgaaaagggccaaccagatgaggagctcaagcccaagaagaaagtcttcgagaagttgcaggctgacttcaaaatttctgaggagtgcatcgcacagtggaagcaaaccaacttcatgaccaagctgggctccatttcctgtaaatcgctgaaaggggggaacattagctag

SEQ ID NO: 33 - DNA, 1935 bp, Homo sapiens
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaaattttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccccacagatgttgtggttgcagtattccccaaaccactgataactcgatttgtccgaatcaagcctgcaacttgggaaactggcatatctatgagatttgaagtatacggttgcaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcaggtatcaaataa

SEQ ID NO: 34 - DNA, 796 bp, Homo sapiens
tcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgaaaactggttcaacatggaaatgattttcattgattcgtatgccagctcacctttttatgatctgccatttcaaagactcatgtttctgctatgaccatgacacgatttaaatcttttcaaatgtttttacgagtattaatcaacattgtattcagctcttaaggcactagtcccttacagaggaccatgctgactgatccattatctatttaaatatttttaaaatattatttatttaactatttataaaacaacttatttttgttcatattatgtcatgtgcacctttgcacagtggttaatgtaataaaatgtgttctttgtatttggtatatttattttgtgttgttcattgaacttttgctatggaacttttgtacttgtttattctttaaaatgaaattccaagcctaattgtgcaacctgattacagaataactggtacacttcatttatccatcaatattatattcaagatataagtaaaaataaactttctgtaaaccagttg

SEQ ID NO: 35 - DNA, 4071 bp, Homo sapiens
atgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaa

(19) United States
(12) Patent Application Publication
(10) Pub. No.: US 2006/0193830 A1
(43) Pub. Date: Aug. 31, 2006

(54) Raav vector compositions and methods for the treatment of choroidal neovascularization

(76) Inventors: William W Hauswirth, Gainesville, FL (US); Peter A Campichiaro, Baltimore, MD (US); Kenneth I Berns, Gainsville, FL (US)

Correspondence Address: HAYNES AND BOONE, LLP, 901 MAIN STREET, SUITE 3100, DALLAS, TX, 75202, US

(21) Appl. No.: 10/508,580

(22) PCT Filed: Mar. 20, 2003
(86) PCT No.: PCT/US03/08667

§ 371 Date: Aug. 24, 2005

(30) Foreign Application Priority Data
Mar. 20, 2002 (US) 60366114

(51) Int. Cl. A61K 48/00; C12N 15/861

(52) U.S. Cl. 424/93.2; 435/456

(57) ABSTRACT

Disclosed are methods for the use of therapeutic polypeptide-encoding polynucleotides in the creation of transformed host cells and transgenic animals is disclosed. In particular, the use of recombinant adeno-associated viral (rAAV) vector compositions comprising polynucleotide sequences that express one or more mammalian PEDF or anti-angiogenesis polypeptides is described. In particular, the invention provides gene therapy methods for the prevention, long-term treatment and/or amelioration of symptoms of a variety of conditions and disorders in a mammalian eye, including, for example blindness, loss of vision, retinal degeneration, macular degeneration, and related disorders resulting from retinal or choroidal neovascularization in affected individuals.

The present application claims priority from provisional application Ser. No. 60/366,114 filed Mar. 20, 2002, the entire contents of which is specifically incorporated herein by reference in its entirety.

The United States government has certain rights in the present invention pursuant to grant numbers EY05951, EY12609, EY11123, EY13101, NS36302, EY07132, EY1765 and EY08571, all from the National Institutes of Health.

### 1. BACKGROUND OF THE INVENTION

1.1 Field of the Invention

The present invention relates generally to the fields of molecular biology and virology, and in particular, to recombinant adeno-associated viral (rAAV) vector compositions comprising nucleic acid segments encoding therapeutic gene products, and their use in the manufacture of medicaments for treating various disorders of the eye including, for example, retinal, ocular or choroidal neovascularization (CNV). Methods and compositions are provided for preparing rAAV-based vector constructs that express one or more therapeutic gene(s) for use in viral-based gene therapies of the mammalian eye, and in particular, for the therapy of neovascularization (NV) disorders.

1.2 Description of Related Art

### 1.2.1 Ocular Neovascularization

Ocular neovascularization (ONV) is a major threat to vision and a complicating feature of many eye diseases. In fact, CNV complicating age-related macular degeneration (AMD) is the most common cause of severe visual loss in people over 60 in developed countries (The Macular Photocoagulation Study Group, 1991). At best, current treatments merely delay severe vision loss, because they are directed at destroying new vessels and do not address the underlying angiogenic stimuli that frequently cause recurrences.

Currently there are no antiangiogenic treatments available for patients with ONV, but several new approaches hold promise. Orally active drugs that inhibit VEGF receptor kinases cause dramatic inhibition of ONV in mice (Seo et al., 1999; Ozaki et al., 2000; Kwak et al., 2000). However, before this can be applied to patients, extensive safety data are needed to be certain there are no serious side effects from systemic inhibition of angiogenesis. To avoid these concerns, local delivery of several agents is being investigated. Phase I clinical trials testing the safety and tolerability of intraocular injections of an aptomer that binds VEGF or an anti-VEGF antibody have been completed and phase II trials have been developed, and preliminary reports have indicated that inflammation may occur, particularly after injection of the anti-VEGF antibody, but it is not considered a severe enough problem to discontinue these approaches (Guyer et al., 2001; Schwartz et al., 2001). Endogenous proteins are likely to be better tolerated and recently several proteins with purported antiangiogenic activity have been identified (O'Reilly et al., 1994; O'Reilly et al., 1997; O'Reilly et al., 1999; Maione et al., 1990; Good et al., 1990; Dawson et al., 1999), and intraocular injection of each of these alone or in combination could be considered. However, the use of large molecules like aptamers or proteins has a major disadvantage of requiring repeated intraocular injections.

Gene transfer offers an alternative means for local delivery of therapeutic proteins to intraocular tissues. Since the eye is a relatively isolated compartment, intraocular injection of a small fraction of the amount of viral vector used for systemic injections results in transduction of a large number of ocular cells and no transduction of cells outside the eye. Recently, it has been demonstrated that intraocular injection of an expression construct for pigment epithelium-derived factor (PEDF) packaged in an adenoviral vector inhibits ONV in three different mouse models (Mori et al., 2001a). This provides proof of concept for the gene transfer approach of treating ONV, but adenoviral vectors have features that may limit their use in humans, including some evidence of toxicity and decreased transgene expression to low levels over the course of a few months. It is not yet known if repeated intraocular injections of adenoviral vectors can be considered. Prolonged transgene expression with no evidence of toxicity has been demonstrated after intraocular injection of expression constructs packaged in rAAV vectors (Bennett et al., 1999; Lau et al., 2000).

### 1.2.2 Current Therapies for CNV are Ineffective

Current treatments for choroidal neovacularization are ineffective, because they are directed at ablating the new blood vessels, but do not address the underlying angiogenic stimuli. As a result, recurrent neovascularization is extremely common and has a devastating effect on vision. To make treatment methods for neovascularization more effective, it is necessary to develop anti-angiogenic therapy. Using adenoviral vectors, it was shown that two proteins which have previously been shown to inhibit tumor angiogenesis (endostatin and PEDF) also inhibit ONV (Mori et al., 2001a: Mori et al., 2001b). While the preliminary results are encouraging, adenoviral vectors invoke an inflammatory response which could cause damage to retinal cells, and also typically provide a short-duration expression of the therapeutic gene. Thus, the need exists for an effective, long-term gene therapy-based treatment that avoids the adverse effects of these and other systems presently afforded by the prior art. What is particularly lacking in the prior art is a safe and effective, long-term therapy for treatment of diseases and dysfunctions of the mammalian eye, and in particular, treatment of human disorders brought about by ocular and CNV.

### 2. SUMMARY OF THE INVENTION

The present invention overcomes these and other limitations inherent in the prior art by providing new rAAV-based genetic constructs that encode one or more mammalian therapeutic polypeptides for the prevention, treatment, and/or amelioration of various disorders resulting from a deficiency in one or more of such polypeptides. In particular, the invention provides AAV-based genetic constructs encoding one or more mammalian neovascularization inhibitory polypeptides variants, and/or active fragments thereof, for use in the treatment of conditions of the mammalian eye, and in particular, the treatment of retinal diseases, and/or CNV and related ocular disorders. In particular, the inventors have demonstrated that pigment epithelium-derived factor (PEDF), angiostatin, endostatin, thrombospondin, neuropilin-1, interferon-alpha, tyrosyl-tRNA synthetase, tryptophanyl-tRNA synthetase, tissue inhibitor of metalloproteinase-3 (TIMP3), the Exon 6 peptide of VEGF, the Exon 7 peptide of VEGF, and soluble vascular endothelial growth factor (VEGF) receptor (sFLT) polypeptides, and biologically active fragments, peptides, and polypeptides thereof are successful in ameliorating the effects of ocular, retinal, and CNV, and offer new methods for treating these diseases in affected animals.

Likewise, the invention provides genetic constructs that encode one or more therapeutic polypeptides useful in the prevention, treatment or amelioration of various ocular disorders, including for example, loss of vision, blindness, macular degeneration, retinal or ocular dysfunction, and related conditions that manifest from an increase in neovascularization of tissues of the eye, and/or the deficiency or absence of physiologically-normal levels of a neovascularization-inhibitory polypeptide such as PEDF, VEGF, angiostatin, endostatin, KDR, interferon-α, neuropilin-1, thrombospondin, TIMP3, or sFLT polypeptides, and/or biologically-active fragments derived from such polypeptides. In particular, the invention provides long-term cost-effective gene-expression-based therapies to treat and/or ameliorate the symptoms of retinal or CNV in affected mammals, and in particular, humans at risk for developing, diagnosed with, or suffering from, one or more ocular dysfunctions resulting from such neovascularization conditions. The invention also provides recombinant adeno-associated viral vectors, viral particles, host cells, and compositions comprising them for use in therapy and in the preparation of medicaments for treating various forms of retinal and/or CNV in mammalian eyes. The invention also provides methods of making and using such compositions, particularly in methods of treatment of mammalian eyes, and methods for providing therapeutically-effective amounts of neovascularization-inhibitory compounds to such eyes. The efficacy and utility of the present compositions and methods have been demonstrated in suitable approved animal models, and represent key advances in the art of treating neovascularization, and in particular, affords new AAV-based gene therapy methods for providing anti-neovascularization therapeuticums to selected mammalian host cells, tissues, and organs.

### 2.1 rAAV Vector Compositions

In a first embodiment, the invention provides an rAAV vector comprising a polypeptide that comprises at least a first nucleic acid segment that encodes a neovascularization-inhibitory peptide or polypeptide, and in particular, a pigment epithelium-derived factor (PEDF) polypeptide, an angiostatin polypeptide, an endostatin polypeptide, a tissue inhibitor of metalloproteinase-3 (TIMP3) polypeptide, a tyrosyl-tRNA synthetase polypeptide, a tryptophanyl-tRNA synthetase polypeptide, a soluble kinase insert domain receptor (KDR), a soluble neuropilin receptor, the Kringle 1-3 peptide of angiostatin, the Kringle 5 peptide of angiostatin, interferon-alpha (IFN-α), thrombospondin-1, a soluble vascular endothelial growth factor (VEGF) receptor (sFLT) polypeptide, the Exon 6 peptide of VEGF, the Exon 7 peptide of VEGF, or biologically-active choroidal neovascularization-inhibitory fragments of any of these peptides and polypeptides, operably linked to at least a first promoter capable of expressing the nucleic acid segment in a suitable host cell transformed with such a vector. In preferred embodiments, the nucleic acid segment encodes a mammalian, and in particular, a human neovascularization-inhibitory polypeptide, such as for example, a polypeptide selected from the group consisting of a human PEDF polypeptide, a human angiostatin polypeptide, a human endostatin polypeptide, a human TIMP3 polypeptide, a human INF-α polypeptide, a human Exon 6 peptide of VEGF, a human Exon 7 peptide of VEGF, a human Kringle 1-3 angiostatin peptide, a human Kringle 5 angiostatin peptide, a human thrombospondin-1 polypeptide, a human tyrosyl-tRNA synthetase polypeptide, a human tryptophanyl-tRNA synthetase polypeptide or a human soluble vascular endothelial growth factor (VEGF) receptor (sFLT) polypeptide, or a biologically-active neovascularization-inhibitory peptide fragment or variant thereof. Alternatively, the therapeutic constructs of the invention may encompass nucleic acid segments that encode choroidal neovascularization-inhibitory polypeptides of any mammalian origin, such as for example nucleic acids, peptides, and polypeptides of murine, ovine, porcine, bovine, equine, epine, caprine, canine, feline, and/or lupine origin, or may encompass modified or site-specifically mutagenized nucleic acid segments that were initially obtained from one or more mammalian species, and genetically modified to be expressed in human cells such that their choroidal neovascularization-inhibitory activity is retained.

In other preferred embodiments, the preferred nucleic acid segments for use in the practice of the present invention, encodes a mammalian, and in particular, a human angiostatin polypeptide or a biologically active fragment or variant thereof. Particularly preferred angiostatin polypeptides include those that comprise at least one, two, three, or four biologically-active Kringle domains of a mammalian angiostatin polypeptide (the first four Kringle domains of plasminogen). In illustrative embodiments, a polypeptide that comprises Kringle domains 1 to 3 of a human angiostatin polypeptide has been show to possess the desired therapeutic properties of the present invention.

Surprisingly, the inventors have also shown that endostatin, the exon 6 peptide (amino acids 121 to 132) fragment of VEGF polypeptide and the exon 7 peptide (amino acids 22 to 44 plus Cys) fragment of VEGF polypeptide are effective at reducing RNV in vivo, almost to the same extent as a PEDF polypeptide or the first three Kringle region peptide (K1-3) of angiostatin.

The polynucleotides comprised in the vectors and viral particles of the present invention preferably comprise at least a first constitutive or inducible promoter operably linked to the nucleic acid segments disclosed herein. Such promoters may be homologous or heterologous promoters, and may be operatively positioned upstream of the nucleic acid segment encoding the therapeutic polypeptide of interest, such that the expression of the segment is under the control of the promoter. The construct may comprise a single promoter, or alternatively, two or more promoters may be used to facilitate expression of the therapeutic gene sequence. Exemplary promoters useful in the practice of the invention include, but are in no way limited to, those promoter sequences that are operable in mammalian, and in particular, human host cells, tissues, and organs, such as for example, a CMV promoter, a β-actin promoter, a hybrid CMV promoter, a hybrid β-actin promoter, an EF1 promoter, a U1a promoter, a U1b promoter, a Tet-inducible promoter and a VP16-LexA promoter being particularly useful in the practice of the invention. In illustrative embodiments, a polynucleotide encoding a therapeutic polypeptide was placed under the control of the chicken β-actin (CBA) promoter and used to produce therapeutically effective levels of the encoded polypeptide when suitable host cells were transformed with the genetic construct.

The polynucleotides comprised in the vectors and viral particles of the present invention may also further optionally comprise one or more native, synthetic, homologous, heterologous, or hybrid enhancer or 5′ regulatory elements, for example, a CMV enhancer, a synthetic enhancer, or an eye- or retinal-specific enhancer operably linked to the therapeutic polypeptide-encoding nucleic acid segments disclosed herein.

The polynucleotides and nucleic acid segments comprised within the vectors and viral particles of the present invention may also further optionally comprise one or more intron sequences.

The polynucleotides comprised in the vectors and viral particles of the present invention may also further optionally comprise one or more native, synthetic, homologous, heterologous, or hybrid post-transcriptional or 3′ regulatory elements operably positioned relative to the therapeutic polypeptide-encoding nucleic acid segments disclosed herein to provide greater expression, stability, or translation of the encoded polypeptides. One such example is the woodchuck hepatitis virus post-transcriptional regulatory element (WPRE), operably positioned downstream of the therapeutic gene of interest.

In illustrative embodiments, the invention concerns administration of one or more biologically-active neovascularization-inhibitory peptides or polypeptides that comprise an at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100, or more contiguous amino acid sequence from the polypeptide and peptide sequences disclosed in Section 6 hereinbelow and particularly those polypeptides and peptides as recited in any one of SEQ ID NO:1 to SEQ ID NO:18.

Likewise, in additional illustrative embodiments, the invention concerns administration of one or more biologically-active neovascularization-inhibitory peptides or polypeptides that are encoded by a nucleic acid segment that comprises at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200, 300, 400, or 500, or more contiguous nucleic acid residues from the DNA sequences disclosed in Section 6 hereinbelow and particularly those DNA sequences as recited in any one of SEQ ID NO:19 to SEQ ID NO:35.

Exemplary adeno-associated viral vector constructs and polynucleotides of the present invention include those that comprise, consist essentially of, or consist of at least a first nucleic acid segment that encodes a peptide or polypeptide that is at least about 75%, at least about 80%, at least about 85%, at least about 90%, at least about 95%, at least about 96%, at least about 97%, at least about 98%, or at least about 99% identical to the sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, or SEQ ID NO:18, wherein the peptide or polypeptide has neovascularization inhibitory activity when administered to a mammalian eye.

Exemplary polynucleotides of the present invention also include those sequences that comprise, consist essentially of, or consist of at least a first nucleic acid segment that is at least about 75%, at least about 80%, at least about 85%, at least about 90%, at least about 95%, at least about 96%, at least about 97%, at least about 98%, or at least about 99% identical to the nucleic acid sequence of SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, or SEQ ID NO:35, wherein the peptide or polypeptide encoded by the nucleic acid segment has neovascularization inhibitory activity when administered to a mammalian eye.

### 2.2 rAAV Viral Particles and Virions, and Host Cells Comprising Them

Other aspects of the invention concern rAAV particles and virions that comprise the vectors of the present invention, pluralities of such particles and virions, as well as pharmaceutical compositions and host cells that comprise one or more of the rAAV vectors disclosed herein, such as for example pharmaceutical formulations of the rAAV vectors or virions intended for administration to a mammal through suitable means, such as, by intramuscular, intravenous, or direct injection to selected cells, tissues, or organs of the mammal, for example, one or both eyes of the selected mammal. Typically, such compositions will be formulated with pharmaceutically-acceptable excipients, buffers, diluents, adjuvants, or carriers, as described hereinbelow, and may further comprise one or more liposomes, lipids, lipid complexes, microspheres, microparticles, nanospheres, or nanoparticle formulations to facilitate administration to the selected organs, tissues, and cells for which therapy is desired.

Further aspects of the invention include mammalian host cells, and pluralities thereof that comprise one or more of the adeno-associated viral vectors, virions, or viral particles as disclosed herein. Particularly preferred cells are human host cells, and in particular, human eye cells, scleral cells, choroidal cells, or retinal cells.

### 2.3 Therapeutic Kits and Pharmaceutical Compositions

Therapeutic kits for treating or ameliorating the symptoms of retinal or CNV, or other condition resulting from a pigment epithelium-derived factor or angiostatin polypeptide deficiency condition in a mammal are also part of the present invention. Such kits typically comprise one or more of the disclosed AAV vector constructs, virion particles, or therapeutic compositions described herein, and instructions for using the kit.

Another important aspect of the present invention concerns methods of use of the disclosed vectors, virions, compositions, and host cells described herein in the preparation of medicaments for treating or ameliorating the symptoms of retinal or CNV, or other conditions resulting from a pigment epithelium-derived factor polypeptide or angiostatin polypeptide deficiency condition in a mammal. Such methods generally involve administration to a mammal, or human in need thereof, one or more of the disclosed vectors, virions, host cells, or compositions, in an amount and for a time sufficient to treat or ameliorate the symptoms of such a deficiency in the affected mammal. The methods may also encompass prophylactic treatment of animals suspected of having such conditions, or administration of such compositions to those animals at risk for developing such conditions either following diagnosis, or prior to the onset of symptoms. Such symptoms may include, but are not limited to, ocular dysfunction, visual impairment, or blindness in affected animals, or may involve the appearance or increase in retinal or CNV in one or both eyes of the mammal at risk for developing a condition arising from hyperneovascularization, or other conditions which manifest themselves in an increased level of choroidal, retinal, or ocular neovascularization.

Another aspect of the invention concerns compositions that comprise one or more of the disclosed adeno-associated viral vectors, virions, viral particles, and host cells as described herein. Pharmaceutical compositions comprising such are particularly contemplated to be useful in therapy, and particularly in the preparation of medicaments for treating ocular neovascularization, choroidal neovascularization, retinal neovascularization, age-related macular degeneration, visual impairment, ocular dysfunction, loss of vision, retinopathy, or blindness in affected mammals, and humans in particular.

### 2.4 Therapeutic Methods

The invention also provides methods for delivering therapeutically-effective amounts of a choroidal or ocular neovascularization inhibitory polypeptide to a mammal in need thereof. Such methods generally comprise at least the step of providing or administering to such a mammal, one or more of the CNV-inhibitory compositions disclosed herein. For example, the method may involve providing to such a mammal, one or more of the rAAV vectors, virions, viral particles, host cells, or pharmaceutical compositions as described herein. Preferably such providing or such administration will be in an amount and for a time effective to provide a therapeutically-effective amount of one or more of the CNV-inhibitory peptides or polypeptides disclosed herein to selected cells, tissues, or organs of the mammal, and in particular, therapeutically-effective levels to the cells of one or both eyes of the mammal. Such methods may include systemic injection(s) of the therapeuticum, or may even involve direct or indirect administration, injection, or introduction of the therapeutic compositions to particular cells, tissues, or organs of the mammal.

For example, the therapeutic composition may be provided to mammal by ocular injection, intravitreolar injection, retinal injection, or subretinal injection.

The invention also provides methods of treating, ameliorating the symptoms, and reducing the severity of choroidal or ocular neovascularization in an animal. These methods generally involve at least the step of providing to an animal in need thereof, one or more of the rAAV vector compositions disclosed herein in an amount and for a time effective to treat NCV or other related ocular dysfunction in the animal. As described above, such methods may involve systemic injection(s) of the therapeuticum, or may even involve direct or indirect administration, injection, or introduction of the therapeutic compositions to particular cells, tissues, or organs of the animal. The method may involve ocular injection, intravitreolar injection, retinal injection, or subretinal injection of the therapeutic compounds to the eye or eyes of the animal, as may be required.

The invention further concerns the use of the adeno-associated viral vectors, virions, viral particles, host cells, and/or the pharmaceutical compositions disclosed herein in the manufacture of a medicament for treating ocular neovascularization, choroidal neovascularization, age-related macular degeneration, vision loss, visual impairment, or blindness in a mammal. This use may involve systemic or localized injection, infection, or administration to one or more cells, tissues, or organs of the mammal. Such use is particularly contemplated in humans that have, are suspected of having, or at risk for developing one or more ocular dysfunctions such as choroidal or ocular neovascularization.

### 3. BRIEF DESCRIPTION OF THE DRAWINGS

The following drawings form part of the present specification and are included to further demonstrate certain aspects of the present invention. The invention may be better understood by reference to the following description taken in conjunction with the accompanying drawings, in which like reference numerals identify like elements, and in which:

FIG. 1A shows intraocular levels of human pigment epithelium-derived factor (PEDF) 4 or 6 weeks after intraocular injection of control vector or AAV-CBA-PEDF. C57BL/6 mice were given a subretinal (squares) or intravitreous (circles) injection of control vector (shaded) or AAV-CBA-PEDF (open). Four weeks after injection, the mice were sacrificed and PEDF levels were measured in whole eye homogenates as by enzyme-linked immunoabsorbant assay (ELISA). The optical density of the standard concentrations (small circles) were plotted to generate the standard curve. The PEDF levels in eyes injected with control vector were below the limit of detection and the levels in eyes injected with AAV-CBA-PEDF ranged from 20 to 70 ng.

FIG. 1B shows intraocular levels of human pigment epithelium-derived factor (PEDF) 4 or 6 weeks after intraocular injection of control vector or AAV-CBA-PEDF. C57BL/6 mice were given a subretinal (squares) or intravitreous (circles) injection of control vector (shaded) or AAV-CBA-PEDF (open). Six weeks after injection, the mice were sacrificed and PEDF levels were measured in whole eye homogenates by ELISA. The PEDF levels in eyes injected with control vector were below the limit of detection and the levels in eyes injected with AAV-CBA-PEDF ranged from 6 to 30 ng.

FIG. 2A and FIG. 2B show AAV-vectored pigment epithelium-derived growth factor (PEDF) inhibits CNV. Four weeks after intravitreous (IV) or subretinal (SR) injection of 4.0×10⁹ particles of control vector (UF12) or 1.5×10⁹ particles of AAV-CBA-PEDF (FIG. 2A) or six weeks after IV or SR injection of 2.4×10⁹ particles of UF12 or 2.0×10¹⁰ particles of AAV-CBA-PEDF (FIG. 2B), C57BL/6 mice had laser-induced rupture of Bruch's membrane at 3 sites in each eye. Two weeks later, the mice were perfused with fluorescein-labeled dextran, choroidal flat mounts were prepared, and the area of CNV at each rupture site was measured by image analysis. *p<0.05 for difference from control vector given by same route by unpaired t-test for populations with unequal variances.

FIG. 3 shows data revealing that endostatin, VEGF Exon 6 Peptide (amino acids 121 to 132 of VEGF), and VEGF Exon 7 Peptide (amino acids 1 to 21 of VEGF) are effective in vivo at reducing retinal NV approximately to the same extent as PEDF and the K1-3 Kringle domains of angiostatin.

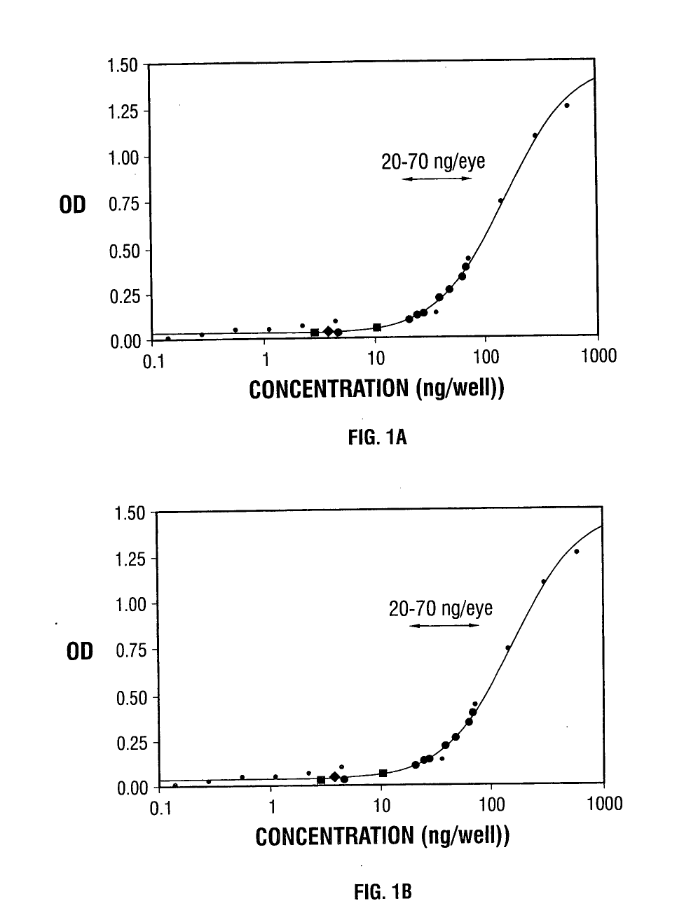

### 4. DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

### 4.1 PEDF Inhibits RNV

A recent study has demonstrated that systemic administration of recombinant PEDF protein inhibits RNV in the murine model of oxygen-induced ischemic retinopathy (Stellmach et al., 2001). In that study, the minimum effective dose of PEDF protein was about 5 μg given by daily intraperitoneal injections. Assuming that 5 μg was the steady-state, whole animal level and correcting for the fractional volume of the eye relative to the whole body (both conservative assumptions), the presumptive threshold therapeutic level of PEDF necessary to inhibit RNV is estimated at about 2 ng/eye.

### 4.2 Promoters and Enhancers

Recombinant AAV vectors form important aspects of the present invention. The term “expression vector or construct” means any type of genetic construct containing a nucleic acid in which part or all of the nucleic acid encoding sequence is capable of being transcribed. In preferred embodiments, expression of the nucleic acid segment occurs in the selected host cells, organs, or tissues, such that the encoded therapeutic peptide or polypeptide of interest (for example, a biologically-active, CNV-inhibitory PEDF polypeptide, angiostatin polypeptide, endostatin polypeptide, TIMP3 polypeptide, tyrosyl-tRNA synthetase polypeptide, tryptophanyl-tRNA synthetase polypeptide, KDR polypeptide, soluble neuropilin receptor polypeptide, IFN-α polypeptide, thrombospondin-1 polypeptide, sFLT polypeptide, or the Kringle 1-3 peptide of angiostatin, the Kringle 5 peptide of angiostatin the Exon 6 peptide of VEGF, or the Exon 7 peptide of VEGF, or biologically-active choroidal neovascularization-inhibitory fragments of any of these peptides and polypeptides) is produced as a result of expression of the gene and subsequent translation of the mRNA into mature protein or peptide.

Particularly useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene. “Upstream” is understood to mean an element is placed 5′ of the reference nucleic acid segment. For example, promoters and enhancers are typically positioned upstream (5′) of the nucleic acid segment encoding the therapeutic polypeptide(s) of interest. Likewise, “downstream” is understood to mean an element is placed 3′ of the nucleic acid segment in question. For example, post-transcriptional regulatory elements (such as the WPRE) are typically positioned downstream (3′) of the nucleic acid segment encoding the therapeutic polypeptide(s) of interest.

In preferred embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with a particular therapeutic gene in its natural environment. Such promoters may include promoters normally associated with other genes, and/or promoters isolated from any bacterial, viral, eukaryotic, or mammalian cell. For example, a CBA promoter operably linked to a human PEDF-encoding nucleic acid segment is a “heterologous” promoter-driven DNA construct. Likewise, when a human angiostatin-encoding DNA sequence is operably positioned under the control of a CMV enhancer, this is referred to as a heterologous enhancer element.

Naturally, it will be desirable in the practice of the invention to employ promoter(s), enhancer(s), and post-transcriptional regulatory element(s) that effectively direct the expression of the sFLT-, endostatin-, INF-α-, thrombospondin-, neuropilin-, KDR-, TIMP3-, PEDF-VEGF-, or angiostatin-encoding nucleic acid segment in the cell type, tissue, organ, or even animal, chosen for expression. The selection of effective promoters and/or enhancers to be used to express selected nucleic acid segments in various cell types, tissues, organs, and animals, to achieve protein expression is generally known to those of skill in the art of molecular biology, for example, see Sambrook et al. (1989), incorporated herein by reference. The promoters, enhancers, and regulatory elements employed in the practice of the invention may be selected to direct expression of the introduced DNA segment under the appropriate conditions in the chosen cell types. As an illustrative example, in human cells, the use of an rAAV vector comprising a CBA promoter and a WPRE operably linked to the therapeutic gene of interest is contemplated to provide the desired therapeutic levels of the encoded protein.

At least one module in a promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the neovascularization-inhibitory polypeptide-encoding nucleic acid segment in the selected or targeted cell. Thus, where a human cell is targeted, it is preferable to position the nucleic acid coding region adjacent to and under the control of a promoter that is capable of being expressed in a human cell. Generally speaking, such a promoter might include either a mammalian or viral promoter, such as a CBA, CMV or an HSV promoter. In certain aspects of the invention tetracycline controlled promoters are also contemplated to be useful.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters that are well known in the art to achieve expression of a transgene is contemplated as well, provided that the levels of expression are sufficient for a given purpose. Tables 1 and 2 below list several elements/promoters that may be employed, in the context of the present invention, to regulate the expression of the present neovascularization-inhibitory polypeptide-encoding nucleic acid segments comprised within the rAAV vectors of the present invention. This list is not intended to be exhaustive of all the possible elements involved in the promotion of transgene expression, but merely to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 1
PROMOTER AND ENHANCER ELEMENTS

| PROMOTER/ | |
|---|---|
| ENHANCER | REFERENCES |
| Immunoglobulin | Banerji et al., 1983; Gilles et al., 1983; |
| Heavy Chain | Grosschedl and Baltimore, 1985; Atchinson |
| | and Perry, 1986, 1987; Imler et al., 1987; |
| | Weinberger et al., 1984; Kiledjian et al., |
| | 1988; Porton et al.; 1990 |
| Immunoglobulin | Queen and Baltimore, 1983; Picard and |
| Light Chain | Schaffner, 1984 |
| T-Cell Receptor | Luria et al., 1987; Winoto and Baltimore, |
| | 1989; Redondo et al.; 1990 |
| HLA DQ a and DQ β | Sullivan and Peterlin, 1987 |
| β-Interferon | Goodbourn et al., 1986; Fujita et al., |
| | 1987; Goodbourn and Maniatis, 1988 |
| Interleukin-2 | Greene et al., 1989 |
| Interleukin-2 | Greene et al., 1989; Lin et al., 1990 |
| Receptor | |
| MHC Class II 5 | Koch et al., 1989 |
| MHC Class II | Sherman et al., 1989 |
| HLA-Dra | |
| β-Actin | Kawamoto et al., 1988; Ng et al.; 1989 |
| Muscle Creatine | Jaynes et al., 1988; Horlick and Benfield, |
| Kinase | 1989; Johnson et al., 1989 |
| Prealbumin | Costa et al., 1988 |
| (Transthyretin) | |
| Elastase I | Omitz et al., 1987 |
| Metallothionein | Karin et al., 1987; Culotta and Hamer, |
| | 1989 |
| Collagenase | Pinkert et al., 1987; Angel et al., 1987 |
| Albumin Gene | Pinkert et al., 1987; Tronche et al., 1989, |
| | 1990 |
| α-Fetoprotein | Godbout et al., 1988; Campere and Tilghman, |
| | 1989 |
| t-Globin | Bodine and Ley, 1987; Perez-Stable and |
| | Constantini, 1990 |
| β-Globin | Trudel and Constantini, 1987 |
| e-fos | Cohen et al., 1987 |
| c-HA-ras | Triesman, 1986; Deschamps et al., 1985 |
| Insulin | Edlund et al., 1985 |
| Neural Cell | Hirsh et al., 1990 |
| Adhesion Molecule | |
| (NCAM) | |
| α_{1-Antitrypain} | Latimer et al., 1990 |
| H2B (TH2B) Histone | Hwang et al., 1990 |
| Mouse or Type | Ripe et al., 1989 |
| I Collagen | |
| Glucose-Regu- | Chang et al., 1989 |
| lated Proteins | |
| (GRP94 and GRP78) | |
| Rat Growth Hormone | Larsen et al., 1986 |
| Human Serum | Edbrooke et al., 1989 |
| Amyloid A (SAA) | |
| Troponin I (TN I) | Yutzey et al., 1989 |
| Platelet-Derived | Pech et al., 1989 |
| Growth Factor | |
| Duchenne Muscular | Klamut et al., 1990 |
| Dystrophy | |
| SV40 | Banerji et al., 1981; Moreau et al., 1981; |
| | Sleigh and Lockett, 1985; Firak and Subra- |
| | manian, 1986; Herr and Clarke, 1986; Imbra |
| | and Karin, 1986; Kadesch and Berg, 1986; |
| | Wang and Calame, 1986; Ondek et al., 1987; |
| | Kuhl et al., 1987; Schaffher et al., 1988 |
| Polyoma | Swartzendruber and Lehman, 1975; Vasseur |
| | et al., 1980; Katinka et al., 1980, 1981; |
| | Tyndell et al., 1981; Dandolo et al., 1983; |
| | de Villiers et al., 1984; Hen et al., 1986; |
| | Satake et al., 1988; Campbell and |
| | Villarreal, 1988 |
| Retroviruses | Kriegler and Botchan, 1982, 1983; Levinson |
| | et al., 1982; Kriegler et al., 1983, 1984a, |
| | b, 1988; Bosze et al., 1986; Miksicek et |
| | al., 1986; Celander and Haseltine, 1987; |
| | Thiesen et al., 1988; Celander et al., |
| | 1988; Chol et al., 1988; Reisman and Rotter, |
| | 1989 |
| Papilloma Virus | Campo et al., 1983; Lusky et al., 1983; |
| | Spandidos and Wilkie, 1983; Spalholz et |
| | al., 1985; Lusky and Botchan, 1986; Cripe |
| | et al., 1987; Gloss et al., 1987; Hirochika |
| | et al., 1987; Stephens and Hentschel, 1987 |
| Hepatitis B | Bulla and Siddiqui, 1986; Jameel and |
| Virus | Siddiqui, 1986; Shaul and Ben-Levy, 1987; |
| | Spandau and Lee, 1988; Vannice and Levinson, |
| | 1988 |
| Human Immuno- | Muesing et al., 1987; Hauber and Cullan, |
| deficiency Virus | 1988; Jakobovits et al., 1988; Feng and |
| | Holland, 1988; Takebe et al., 1988; Rosen |
| | et al., 1988; Berkhout et al., 1989; Laspia |
| | et al., 1989; Sharp and Marciniak, 1989; |
| | Braddock et al., 1989 |
| Cytomegalovirus | Weber et al., 1984; Boshart et al., 1985; |
| | Foecking and Hofstetter, 1986 |
| Gibbon Ape | Holbrook et al., 1987; Quinn et al., 1989 |
| Leukemia Virus | |

TABLE 2
INDUCIBLE ELEMENTS

| ELEMENT | INDUCER | REFERENCES |
|---|---|---|
| MT II | Phorbol | Palmiter et al., 1982; Haslinger |
| | Ester (TFA) | and Karin, 1985; Searle et al., |
| | Heavy metals | 1985; Stuart et al., 1985; |
| | | Imagawa et al., 1987, Karin |
| | | et al., 1987; Angel et al., |
| | | 1987b; McNeall et al., 1989 |
| MMTV (mouse | Glucocor- | Huang et al., 1981; Lee et al., |
| mammary tumor | ticoids | 1981; Majors and Varmus, |
| virus) | | 1983; Chandler et al., 1983; |
| | | Lee et al., 1984; Ponta et al., |
| | | 1985; Sakai et al., 1988 |
| β-Interferon | poly(rI)x | Tavernier et al., 1983 |
| | poly(rc) | |
| Adenovirus | Ela | Imperiale and Nevins, 1984 |
| 5 E2 | | |
| Collagenase | Phorbol | Angel et al., 1987a |
| | Ester (TPA) | |
| Stromelysin | Phorbol | Angel et al., 1987b |
| | Ester (TPA) | |
| SV40 | Phorbol | Angel et al., 1987b |
| | Ester (TPA) | |
| Murine MX | Interferon, | |
| Gene | Newcastle | |
| | Disease | |
| | Virus | |
| GRP78 Gene | A23187 | Resendez et al., 1988 |
| α-2-Macro- | IL-6 | Kunz et al., 1989 |
| globulin | | |
| Vimentin | Serum | Rittling et al., 1989 |
| MHC Class I | Interferon | Blanar et al., 1989 |
| Gene H-2κb | | |
| HSP70 | Ela, SV40 | Taylor et al., 1989; Taylor and |
| | Large T | Kingston, 1990a, b |
| | Antigen | |
| Proliferin | Phorbol | Mordacq and Linzer, 1989 |
| | Ester-TPA | |
| Tumor | FMA | Hensel et al., 1989 |
| Necrosis | | |
| Factor | | |
| Thyroid | Thyroid | Chatterjee et al., 1989 |
| Stimulating | Hormone | |
| Hormone a | | |
| Gene | | |

As used herein, the terms “engineered” and “recombinant” cells are intended to refer to a cell into which an exogenous DNA segment, such as DNA segment that leads to the transcription of a neovascularization-inhibitory polypeptide or a ribozyme specific for such a polypeptide product, has been introduced. Therefore, engineered cells are distinguishable from naturally occurring cells, which do not contain a recombinantly introduced exogenous DNA segment. Engineered cells are thus cells having DNA segment introduced through the hand of man.

To express a CNV-inhibitory peptide-encoding nucleic acid segment in accordance with the present invention one would prepare an rAAV expression vector that comprises a CNV-inhibitory peptide- or polypeptide-encoding nucleic acid segment under the control of one or more promoters. To bring a sequence “under the control of” a promoter, one positions the 5′ end of the transcription initiation site of the transcriptional reading frame generally between about 1 and about 50 nucleotides “downstream” of (i.e., 3′ of) the chosen promoter. The “upstream” promoter stimulates transcription of the DNA and promotes expression of the encoded polypeptide. This is the meaning of “recombinant expression” in this context. Particularly preferred recombinant vector constructs are those that comprise an rAAV vector. Such vectors are described in detail herein.

### 4.3 Pharmaceutical Compositions

In certain embodiments, the present invention concerns formulation of one or more of the rAAV compositions disclosed herein in pharmaceutically acceptable solutions for administration to a cell or an animal, either alone or in combination with one or more other modalities of therapy, and in particular, for therapy of the mammalian eye and tissues thereof.

It will also be understood that, if desired, nucleic acid segments, RNA, DNA or PNA compositions that express one or more of the neovascularization-inhibitory therapeutic gene products as disclosed herein may be administered in combination with other agents as well, such as, e.g., proteins or polypeptides or various pharmaceutically-active agents, including one or more systemic or topical administrations of neovascularization-inhibitory polypeptides, biologically active fragments, or variants thereof. In fact, there is virtually no limit to other components that may also be included, given that the additional agents do not cause a significant adverse effect upon contact with the target cells or host tissues. The rAAV compositions may thus be delivered along with various other agents as required in the particular instance. Such compositions may be purified from host cells or other biological sources, or alternatively may be chemically synthesized as described herein. Likewise, such compositions may further comprise substituted or derivatized RNA, DNA, or PNA compositions.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound(s) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the AAV vector-based therapeutic constructs in suitably formulated pharmaceutical compositions disclosed herein either subcutaneously, intraocularly, intravitreally, parenterally, intravenously, intramuscularly, intrathecally, or even orally, intraperitoneally, or by nasal inhalation, including those modalities as described in U.S. Pat. No. 5,543,158; U.S. Pat. No. 5,641,515 and U.S. Pat. No. 5,399,363 (each specifically incorporated herein by reference in its entirety). Solutions of the active compounds as freebase or pharmacologically acceptable salts may be prepared in sterile water and may also suitably mixed with one or more surfactants, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active AAV vector-delivered neovascularization-inhibitory polypeptide-encoding polynucleotides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The AAV vector compositions disclosed herein may also be formulated in a neutral or salt form. Pharmaceutically-acceptable salts include the acid addition salts (formed with the free amino groups of the protein), which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human, and in particular, when administered to the cells, and tissues of the human eye. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

### 4.4 Liposome-, Nanocapsule-, and Microparticle-Mediated Delivery

In certain embodiments, the inventors contemplate the use of liposomes, nanocapsules, microparticles, microspheres, lipid particles, vesicles, and the like, for the introduction of the compositions of the present invention into suitable host cells. In particular, the rAAV vectors, virions, viral particles, or pharmaceutically-acceptable compositions of the present invention may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon and Papahadjopoulos, 1988; Allen and Choun, 1987; U.S. Pat. No. 5,741,516, specifically incorporated herein by reference in its entirety). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally resistant to transfection by other procedures including T cell suspensions, primary hepatocyte cultures and PC 12 cells (Renneisen et al., 1990; Muller et al., 1990). In addition, liposomes are free of the DNA length constraints that are typical of viral-based delivery systems. Liposomes have been used effectively to introduce genes, drugs (Heath and Martin, 1986; Heath et al., 1986; Balazsovits et al., 1989; Fresta and Puglisi, 1996), radiotherapeutic agents (Pikul et al., 1987), enzymes (Imaizumi et al., 1990a; Imaizumi et al., 1990b), viruses (Faller and Baltimore, 1984), transcription factors and allosteric effectors (Nicolau and Gersonde, 1979) into a variety of cultured cell lines and animals. In addition, several successful clinical trails examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses, toxicity or gonadal localization after systemic delivery (Mori and Fukatsu, 1992).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

In addition to the teachings of Couvreur et al. (1977; 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

In addition to temperature, exposure to proteins can alter the permeability of liposomes. Certain soluble proteins, such as cytochrome c, bind, deform and penetrate the bilayer, thereby causing changes in permeability. Cholesterol inhibits this penetration of proteins, apparently by packing the phospholipids more tightly. It is contemplated that the most useful liposome formations for antibiotic and inhibitor delivery will contain cholesterol.

The ability to trap solutes varies between different types of liposomes. For example, MLVs are moderately efficient at trapping solutes, but SUVs are extremely inefficient. SUVs offer the advantage of homogeneity and reproducibility in size distribution, however, and a compromise between size and trapping efficiency is offered by large unilamellar vesicles (LUVs). These are prepared by ether evaporation and are three to four times more efficient at solute entrapment than MLVs.

In addition to liposome characteristics, an important determinant in entrapping compounds is the physicochemical properties of the compound itself. Polar compounds are trapped in the aqueous spaces and nonpolar compounds bind to the lipid bilayer of the vesicle. Polar compounds are released through permeation or when the bilayer is broken, but nonpolar compounds remain affiliated with the bilayer unless it is disrupted by temperature or exposure to lipoproteins. Both types show maximum efflux rates at the phase transition temperature.

Liposomes interact with cells via four different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. It often is difficult to determine which mechanism is operative and more than one may operate at the same time.

The fate and disposition of intravenously injected liposomes depend on their physical properties, such as size, fluidity, and surface charge. They may persist in tissues for h or days, depending on their composition, and half lives in the blood range from min to several h. Larger liposomes, such as MLVs and LUVs, are taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominate site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow, and lymphoid organs.

Targeting is generally not a limitation in terms of the present invention. However, should specific targeting be desired, methods are available for this to be accomplished. Antibodies may be used to bind to the liposome surface and to direct the antibody and its drug contents to specific antigenic receptors located on a particular cell-type surface. Carbohydrate determinants (glycoprotein or glycolipid cell-surface components that play a role in cell-cell recognition, interaction and adhesion) may also be used as recognition sites as they have potential in directing liposomes to particular cell types. Mostly, it is contemplated that intravenous injection of liposomal preparations would be used, but other routes of administration are also conceivable.

Alternatively, the invention provides for pharmaceutically acceptable nanocapsule formulations of the AAV vector-based polynucleotide compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; Couvreur, 1988; zur Muhlen et al., 1998; Zambaux et al. 1998; Pinto-Alphandry et al., 1995 and U.S. Pat. No. 5,145,684, specifically incorporated herein by reference in its entirety).

### 4.5 Additional Modes of Delivery

In addition to the methods of delivery described above, the following techniques are also contemplated as alternative methods of delivering the disclosed rAAV vector based polynucleotide compositions to a target cell or particular animal, organ, or tissue. Sonophoresis (i.e., ultrasound) has been used and described in U.S. Pat. No. 5,656,016 (specifically incorporated herein by reference in its entirety) as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. No. 5,770,219 and U.S. Pat. No. 5,783,208) and feedback-controlled delivery (U.S. Pat. No. 5,697,899), each specifically incorporated herein by reference in its entirety.

### 4.6 Therapeutic and Diagnostic Kits

The invention also encompasses one or more compositions together with one or more pharmaceutically-acceptable excipients, carriers, diluents, adjuvants, and/or other components, as may be employed in the formulation of particular rAAV-polynucleotide delivery formulations, and in the preparation of therapeutic agents for administration to a mammal, and in particularly, to a human, for one or more of the ocular diseases and dysfunctions described herein. In particular, such kits may comprise one or more of the disclosed rAAV compositions in combination with instructions for using the viral vector in the treatment of such RNV, CNV, and ONV disorders in a mammal, and may typically further include containers prepared for convenient commercial packaging.

As such, preferred animals for administration of the pharmaceutical compositions disclosed herein include mammals, and particularly humans. Other preferred animals include animals of commercial interest, domesticated livestock, and household pets such as dogs and cats under the care of veterinary professionals. The composition may include partially or significantly purified rAAV vectors or viral compositions, either alone, or in combination with one or more additional active ingredients, which may be obtained from natural or recombinant sources, or which may be obtainable naturally or either chemically synthesized, or alternatively produced in vitro from recombinant host cells expressing DNA segments encoding such additional active ingredients.

Therapeutic kits may also be prepared that comprise at least one of the rAAV vector-based gene therapy compositions disclosed herein and instructions for using the composition as a therapeutic agent. The container means for such kits may typically comprise at least one vial, test tube, flask, bottle, syringe or other container means, into which the disclosed rAAV composition(s) may be placed, and preferably suitably aliquoted. Where a neovascularization-inhibitory composition is also provided, the kit may also contain a second distinct container means into which this second composition may be placed. Alternatively, the plurality of neovascularization-inhibitory biologically-active compositions may be prepared in a single pharmaceutical composition, and may be packaged in a single container means, such as a vial, flask, syringe, bottle, or other suitable single container means. The kits of the present invention may also include one or more means for containing the vial(s) or syringes in close confinement for commercial sale, such as, e.g., injection or blow-molded plastic containers into which the desired vial(s) or syringes are retained.

### 4.7 Adeno-Associated Virus (AAV)

AAV is particularly attractive for gene transfer because it does not induce any pathogenic response and can integrate into the host cellular chromosome (Kotin et al., 1990). The AAV terminal repeats (TRs) are the only essential cis-components for the chromosomal integration (Muzyczka and McLaughin, 1988). These TRs are reported to have promoter activity (Flotte et al., 1993). They may promote efficient gene transfer from the cytoplasm to the nucleus or increase the stability of plasmid DNA and enable longer-lasting gene expression (Bartlett et al., 1996). Studies using recombinant plasmid DNAs containing AAV TRs have attracted considerable interest. AAV-based plasmids have been shown to drive higher and longer transgene expression than the identical plasmids lacking the TRs of AAV in most cell types (Philip et al., 1994; Shafron et al., 1998; Wang et al., 1999).

AAV (Ridgeway, 1988; Hermonat and Muzyczka, 1984) is a parovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. Five serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter (Muzyczka and McLaughlin, 1988).

The AAV DNA is approximately 4.7 kilobases long. It contains two open reading frames and is flanked by two ITRs. There are two major genes in the AAV genome: rep and cap. The rep gene encodes a protein responsible for viral replications, whereas the cap gene encodes the capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three viral promoters have been identified and named p5, p19, and p40, according to their map position. Transcription from p5 and p 19 results in production of rep proteins, and transcription from p40 produces the capsid proteins (Hermonat and Muzyczka, 1984).

There are several factors that prompted researchers to study the possibility of using rAAV as an expression vector. One is that the requirements for delivering a gene to integrate into the host chromosome are surprisingly few. It is necessary to have the 145-bp ITRs, which are only 6% of the AAV genome. This leaves room in the vector to assemble a 4.5-kb DNA insertion. While this carrying capacity may prevent the AAV from delivering large genes, it is amply suited for delivering the antisense constructs of the present invention.

AAV is also a good choice of delivery vehicles due to its safety. There is a relatively complicated rescue mechanism: not only wild type adenovirus but also AAV genes are required to mobilize rAAV. Likewise, AAV is not pathogenic and not associated with any disease. The removal of viral coding sequences minimizes immune reactions to viral gene expression, and therefore, rAAV does not evoke an inflammatory response. AAV therefore, represents an ideal candidate for delivery of the present hammerhead ribozyme constructs.

### 5. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### 5.1 Example 1

### AAV-Mediated Expression of PEDF or Angiostatin (K1K3) Reduces RNV in a Mouse Model of Ischemic Retinopathy

The present example describes methods for the rAAV-mediated expression of pigment epithelium-derived factor (PEDF) polypeptides or the biologically-active peptide fragment that comprises Kringle domains 1-3 (K1-3 or K1K3) of an angiostatin polypeptide in reducing aberrant microvessel formation in a mouse model of ischemia-induced neonatal retinal NV.

### 5.1.1 Methods

rAAV vectors expressing the therapeutic genes of interest were injected into one eye of Day 0 (P0) newborn mouse pups. Retinal NV was induced in P7 mice exposed to 73%±2% oxygen for 5 days, followed by room air for another 5 days. Retinal NV was quantified by the number of endothelial cell nuclei internal to the inner limiting membrane in P17 eye sections. Protein levels for expressed PEDF and K1K3 were measured by indirect sandwich ELISA for the time frame corresponding to the ischemia-induced model.

### 5.1.2 Results

The number of endothelial cell nuclei internal to the inner limiting membrane in eyes treated with rAAV-PEDF or rAAV-K1K3 was reduced on average by 30-40% compared to control eyes. The protein levels measured by ELISA indicate expression of PEDF or K1K3 is detectable as early as 1 day post-injection and persists for the period of the experimental model at therapeutic levels.

Expression of either PEDF or K1K3 from rAAV vectors reduces the level of retinal NV in this model of ischemic retinopathy. The protein levels detected by ELISA correlate well with the reduction in NV and confirm that short-term expression from rAAV vectors is a viable therapeutic method. The immediate-early expression pattern appears to be due, at least in part, to the rapidly dividing nature of retinal cells at this developmental stage.

### 5.2 Example 2

### AAV-Mediated Gene Transfer of Pigment Epithelium-Derived Factor Inhibits CNV

rAAV vectors have been used to express several different proteins in the eye. This example demonstrates that AAV-mediated intraocular gene transfer of pigment epithelium-derived factor (PEDF) inhibits the development of CNV in a murine model.

C57BL/6 mice were given intravitreous or subretinal injections of a PEDF expression construct packaged in an AAV vector (AAV-CBA-PEDF) or control vector (AAV-CBA-GFP). After 4 or 6 weeks, Bruch's membrane was ruptured by laser photocoagulation at three sites in each eye. After 14 days, the area of CNV at each rupture site was measured by image analysis. Intraocular levels of PEDF were measured by enzyme-linked immunoabsorbant assay.

Four to six weeks after intraocular injection of AAV-CBA-PEDF, levels or PEDF in whole eye homogenates were 6-70 ng, significantly above control levels. The average area of CNV at sites of Bruch's membrane rupture showed no significant difference in eyes injected with AAV-CBA-PEDF compared to uninjected eyes. In contrast, 4-6 weeks after intraocular injection of 1.5×10⁹ or 2.0×10¹⁰ particles of AAV-CBA-PEDF, the area of CNV at Bruch's membrane rupture sites was significantly decreased compared to CNV area at rupture sites in eyes injected with AAV-CBA-GFP or uninjected eyes.

These data suggest that intraocular expression of PEDF or other antiangiogenic proteins with AAV vectors provide new treatment approaches for ONV.

### 5.2.1 Materials and Methods

### 5.2.1.1 Production of rAAV Vectors Expressing PEDF

Cloning of human PEDF has been previously described (Mori et al., 2001a). Recombinant AAV constructs were based on the pTR-UF (Zolotukhin et al., 1996), a viral vector plasmid in which an expression cassette, consisting of a CMV enhancer and a truncated CBA promoter-exon 1-intron 1, and a poliovirus internal ribosome entry sequence precede the PEDF cDNA and a SV40 polyadenylation site follows it. The entire construct is flanked by inverted terminal repeat sequences from AAV-2. AAV-CBA-PEDF vector titers were 1.5×10¹² or 2.0×10¹³ particles/ml. The control vector (UF12) was constructed identically except that the PEDF coding region was not inserted. It was used at 2.4×10¹² or 4.0×10¹² particles/ml. Contaminating helper Adenovirus and wild type AAV, assayed by serial dilution cytopathic effects or infectious centers respectively, were lower than our detection limit of six orders of magnitude below recombinant AAV vector titers.

### 5.2.1.2 Mouse Model of Laser-Induced CNV

Adult C57BL/6 mice were given either an intravitreous injection of UF12 or AAV-CBA-PEDF by previously published techniques (Mori et al., 2001a). Intravitreous injections were done with a Harvard pump microinjection apparatus and pulled glass micropipets. Each micropipet was calibrated to deliver 1 μl of vehicle containing the appropriate number of viral particles upon depression of a foot switch. The mice were anesthetized, pupils were dilated, and under a dissecting microscope, the sharpened tip of the micropipet was passed through the sclera just behind the limbus into the vitreous cavity and the foot switch was depressed. Subretinal injections were performed using a condensing lens system on the dissecting microscope, which allowed visualization of the retina during the injection. The pipette tip was passed through the sclera posterior to the limbus and was positioned just above the retina. Depression of the foot switch caused the jet of injection fluid to penetrate the retina. The blebs were quite uniform in size and in each case two of the laser burns were encompassed by the bleb and one was outside the region of the bleb.

Two independent experiments were performed. In the first, mice were given intravitreous or subretinal injection of 1 μl containing 1.5×10⁹ particles of AAV-CBA-PEDF or 4.0×10⁹ particles of control vector, and then 4 weeks after injection, Bruch's membrane was ruptured with laser photocoagulation at three locations in each eye. Some mice were not treated with laser photocoagulation and were sacrifice to measure ocular PEDF levels by ELISA. In the second experiment, mice were given intravitreous or subretinal injection of 1 μl containing 2.4×10⁹ particles of control vector or 2.0×10¹⁰ particles of AAV-CBA-PEDF, and then 6 weeks after injection, Bruch's membrane was ruptured by laser photocoagulation at three sites in each eye as previously described (Tobe et al., 1998). Briefly, laser photocoagulation (532 nm wavelength, 100 μm spot size, 0.1 seconds duration, and 120 mW intensity) was delivered using the slit lamp delivery system and a hand-held cover slide as a contact lens. Burns were performed in the 9, 12, and 3 o'clock positions 2-3 disc diameters from the optic nerve. Production of a vaporization bubble at the time of laser, which indicates rupture of Bruch's membrane, is an important factor in obtaining CNV (Tobe et al., 1998a), so only burns in which a bubble was produced were included in the study.

### 5.2.1.3 Measurement of the Sizes of Laser-Induced CNV Lesions

Two weeks after laser treatment, the sizes of CNV lesions were measured in choroidal flat mounts (Edelman and Castro, 2000). Mice used for the flat mount technique were anesthetized and perfused with 1 ml of phosphate-buffered saline containing 50 mg/ml of fluorescein-labeled dextran (2×10⁶ average mw, Sigma, St. Louis, Mo.) as previously described (Tobe et al., 1998b). The eyes were removed and fixed for 1 hr in 10% phosphate-buffered formalin. The cornea and lens were removed and the entire retina was carefully dissected from the eyecup. Radial cuts (4-7, average 5) were made from the edge to the equator and the eyecup was flat mounted in Aquamount with the sclera facing down. Flat mounts were examined by fluorescence microscopy on an Axioskop microscope (Zeiss, Thornwood, N.Y.) and images were digitized using a 3-color CCD video camera (IK-TU40A, Toshiba, Tokyo, Japan) and a frame grabber. Image-Pro Plus software (Media Cybernetics, Silver Spring, Md.) was used to measure the total area of hyperfluorescence associated with each burn, corresponding to the total fibrovascular scar. The areas within each eye were averaged to give one experimental value and mean values were calculated for each treatment group and compared by Student's unpaired t-test.

Some mice were sacrificed two weeks after laser treatment and eyes were rapidly removed and frozen in optimum cutting temperature embedding compound (OCT; Miles Diagnostics, Elkhart, Ind.). Frozen serial sections (10 μm) were cut through the entire extent of each burn and histochemically stained with biotinylated Griffonia simplicifolia lectin B4 (GSA; Vector Laboratories, Burlingame, Calif.), which selectively binds vascular cells. Slides were incubated in methanol/H₂O₂ for 10 min at 4° C., washed with 0.05 M Tris-buffered saline, pH 7.6 (TBS), and incubated for 30 min in 10% normal porcine serum. Slides were incubated 2 hr at room temperature with biotinylated GSA and, after rinsing with 0.05M TBS, they were incubated with avidin coupled to peroxidase (Vector Laboratories) for 45 min at room temperature. After a 10 min wash in 0.05M TBS, slides were incubated with HistoMark Red (Kirkegaard and Perry, Cabin John, Md.) to give a red reaction product that is distinguishable from melanin and counterstained with Contrast Blue (Kirkegaard and Perry).

### 5.2.1.4 ELISA for PEDF

Mice were sacrificed and eyes were removed and quick frozen in 100 μl of PBS pH 7.4 with 0.05% PMSF and homogenized manually on ice using a ground glass tissue homogenizer followed by three freeze thaw cycles on liquid nitrogen and wet ice. The homogenate was centrifuged in a refrigerated desktop centrifuge to pellet the insoluble material and the supernatant was loaded in sample wells for detection by ELISA. PEDF was detected by a sandwich ELISA procedure using a biotin-conjugated antibody and HRP-conjugated avidin for detection. Rabbit anti-PEDF was coated on 96-well Immulon flat bottom microtiter plates (Thermo Labsystems Oy, Helsinki, Finland) in 0.1 M NaHCO₃ overnight at 4° C. The wells were blocked with 10% fetal bovine serum in PBS pH 7.4 for 2 hr at 37° C. PEDF protein standards and eye extract samples were loaded as 100 μl aliquots into wells and the plate was kept overnight at 4° C. Detection consisted of a secondary mouse polyclonal anti-PEDF followed by a biotin-conjugated rat anti-mouse IgG (ICN Biomedicals, Costa Mesa, Calif.) and HRP conjugated avidin (Pharmingen, San Diego, Calif.). Each step of detection was conducted with plate agitation at room temperature for 1-2 hr and the plate was washed 5 times between steps. TMB peroxidase substrate system (Kierkegaard & Perry) was allowed to reach fully developed color, usually 30 min, before stopping the reaction with 1 M H₃PO₄. The plates were read in an automated microplate reader at 450 nm.

### 5.2.2 Results

### 5.2.2.1 PEDF is Expressed in Mouse Eyes 4 or 6 Weeks After Intravitreous or Subretinal Injection of AAV-CBA-PEDF

Mice given an intravitreous or subretinal injection of AAV-CBA-PEDF showed levels of human PEDF ranging from 20-70 ng/eye 4 weeks after the injection. In a second series of mice, the range of PEDF was 6-30 ng/eye 6 weeks after intravitreous or subretinal injection of vector. All mice given intravitreous or subretinal injections of control vector had undetectable levels of PEDF. Given the variability from injection to injection, these ranges of PEDF are likely to be the same at 4 and 6 weeks postinjection and they are well above background levels observed in control eyes. Subretinal and intravitreous injection of PEDF vector produced similar and overlapping levels of protein expression.

### 5.2.2.2 Intravitreous or Subretinal Injections of CBA-PEDF Reduces Laser-Induced CNV

Representative flat mounts and cross-sections were prepared from the group of mice treated with laser 6 weeks after vector injection. These results showed smaller CNV lesions in eyes injected with AAV-CBA-PEDF compared to eyes injected with control vector. C57BL/6 mice were given an intravitreous or subretinal injection of control vector or AAV-CBA-PEDF. Six weeks after injection, Bruch's membrane was ruptured with laser photocoagulation at 3 sites in each eye. Two weeks after rupture of Bruch's membrane, the mice were perfused with fluorescein-labeled dextran and choroidal flat mounts were prepared or eyes were frozen and serial sections were stained with Griffonia simplicifolia lectin B4, which stains vascular cells, and counterstained with hematoxylin and eosin. Fluorescence microscopy showed: (1) a large CNV lesion at a Bruch's membrane rupture site in an eye that did not receive any injections; (2) a frozen section through the center of a CNV lesion in another uninjected eye showed a large maximum diameter (the lesion was quite thick along the surface of the CNV); (3) a large CNV lesion in an eye that received an intravitreous injection of control vector; (4) a frozen section through the center of a CNV lesion in another eye that received an intravitreous injection of control vector showed a large maximum diameter; (5) a large CNV lesion in an eye that received a subretinal injection of control vector; (6) a frozen section through the center of a CNV lesion in another eye that received a subretinal injection of control vector showed a large maximum diameter; (7) a small area of CNV in an eye that received an intravitreous injection of AAV-CBA-PEDF; (8) a frozen section through the center of a CNV lesion in another eye that received an intravitreous injection of AAV-CBA-PEDF showed a small maximum diameter; (9) a small area of CNV in an eye that received a subretinal injection of AAV-CBA-PEDF; and (10) a frozen section through the center of a CNV lesion in another eye that received a subretinal injection of AAV-CBA-PEDF showed a small maximum diameter.

Mice that did not receive an intraocular injection showed large areas of CNV at sites of rupture of Bruch's membrane. Control mice that received an intravitreous or subretinal injection of 2.4×10⁹ particles of UF12, showed areas of CNV that were very similar to those seen in uninjected mice. Mice that received an intravitreous or subretinal injection of 2×10¹⁰ particles of AAV-CBA-PEDF, showed visibly smaller areas of CNV compared to uninjected mice or mice injected with UF12.

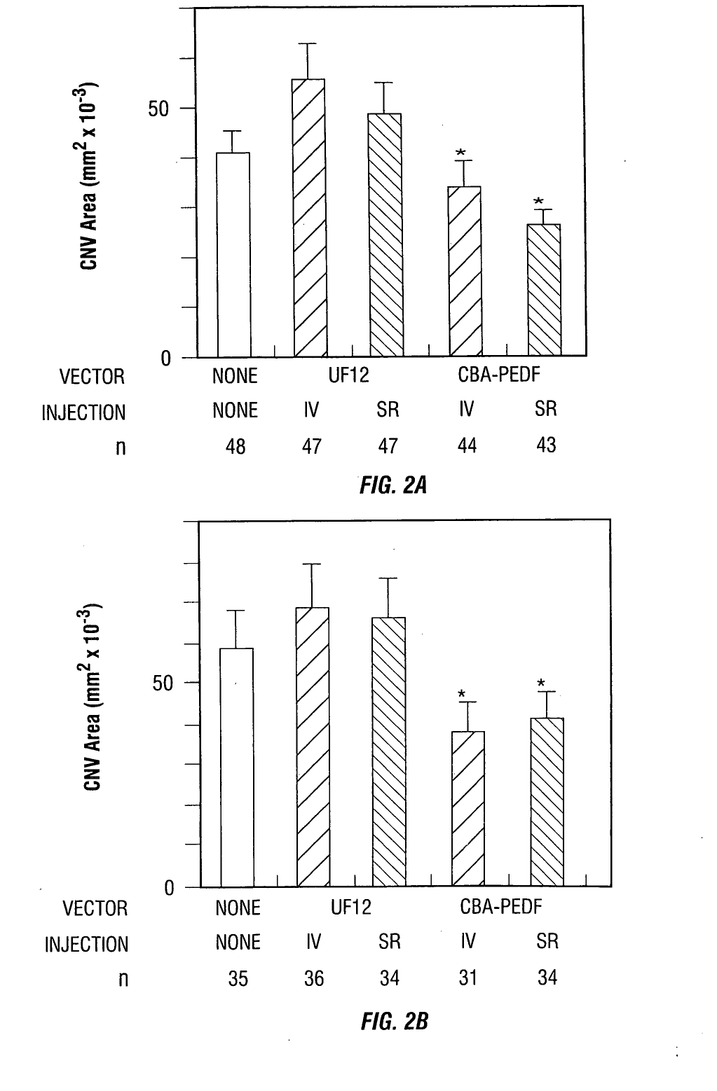

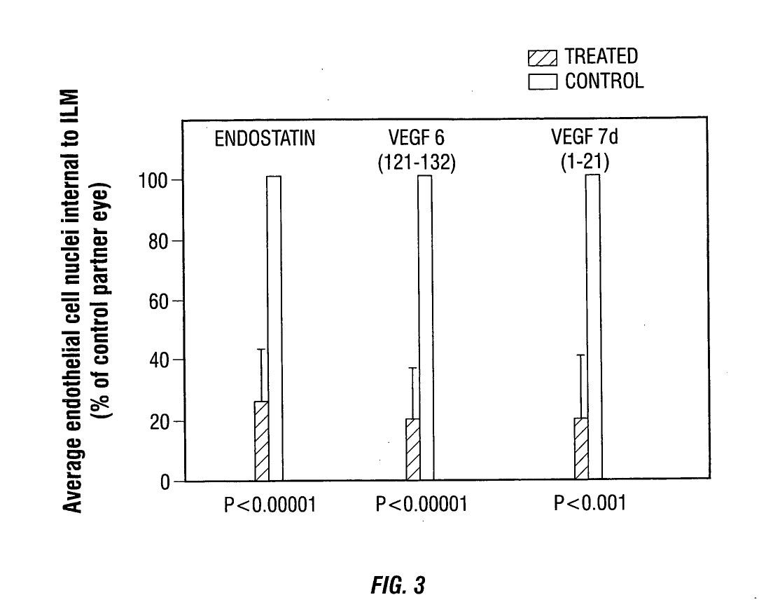

Measurement of the area of CNV by image analysis in each of the groups, showed that there was no significant difference between the mean area in uninjected mice and mice given an intravitreous or subretinal injection of empty virus (FIG. 2A and FIG. 2B). Mice treated with laser 4 (FIG. 2A) or 6 weeks (FIG. 2B) after intravitreous or subretinal injection of AAV-CBA-PEDF showed significantly smaller mean areas of CNV compared to uninjected mice or mice injected with control vector.

The ocular levels of PEDF after gene transfer that resulted in inhibition of CNV are likely to be above the therapeutic level for inhibition of CNV. The demonstration that AAV-mediated intraocular expression of PEDF reduces CNV at sites of rupture of Bruch's membrane is important regarding practical aspects of treatment development. Patients with age-related macular degeneration (AMD) are at risk for the development of CNV for many years and long-term treatment is needed. Prolonged intraocular transgene expression has been achieved with AAV vectors and therefore, they may provide the sustained intraocular production of antiangiogenic proteins that is likely to be needed to counter chronic production of angiogenic stimuli.

The results obtained in the present invention indicate that PEDF is a particularly appealing therapeutic candidate for patients with AMD. While CNV is the major cause of severe visual loss in patients with AMD, most moderate loss of vision is due to death of photoreceptors and retinal pigmented epithelial (RPE) cells. PEDF was first identified as a component of conditioned media of cultured fetal RPE cells that causes neurite outgrowth of Y79 retinoblastoma cells (Tombran-Tink et al., 1991; Steele et al., 1993). Several studies have suggested that PEDF has neuroprotective activity (Taniwaki et al., 1995; Araki et al., 1998; DeCoster et al., 1999; Bilak et al., 1999; Cao et al., 1999; Houenou et al., 1999, including protection of photoreceptors separated from the RPE from degeneration and loss of opsin immunoreactivity (Jablonski et al., 2000). Therefore, long-term AAV-mediated expression of PEDF in the eyes of patients with early AMD may slow progression of the degeneration as well as reduce the likelihood of CNV.

In rodents, AAV-mediated expression of proteins in the eye appears to occur for the entire life of the animal. While such long-term expression is an advantage on one hand, it also raises certain questions about the choice of inducible or constitutive expression of the therapeutic polypeptide delivered using the AAV vectors disclosed herein. For example, if chronic expression of an antiangiogenic agent in the eye has some unsuspected deleterious effect, it may not be possible to halt the expression. Use of promoter systems that allow inducible expression could provide a safety net until the effects of long-term expression of PEDF in the eye are better understood. In any case, the demonstration herein that AAV-mediated expression of PEDF in the eye inhibits CNV is an important step in the development of antiangiogenic gene therapy for patients with AMD.

### 6. ILLUSTRATIVE POLYPEPTIDE AND POLYNUCLEOTIDE SEQUENCES USEFUL IN THE PRACTICE OF THE PRESENT INVENTION

### 6.1 PEDF Polypeptide and Polynucleotide Sequences

### 6.1.1 Human PEDF

(SEQ ID NO:1)

Human PEDF Polypeptide
MQALVLLLCIGALLGHSSCQNPASPPEEGSPDPDSTGALVEEEDPFFKVP
VNKLAAAVSNFGYDLYRVRSSMSPTTNVLLSPLSVATALSALSLGADERT
ESIIHPALYYDLISSPDIHGTYKELLDTVTAPQKNLKSASRIVFEKKLRI
KSSFVAPLEKSYGTRPRVLTGNPRLDLQEINNWVQAQMKGKLARSTKEIP
DEISILLLGVAHFKGQWVTKFDSRKTSLEDFYLDEERTVRVPMMSDPKAV
LRYGLDSDLSCKIAQLPLTGSMSIIFFLPLKVTQNLTLIEESLTSEFIHD
IDRELKTVQAVLTVPKLKLSYEGEVTKSLQEMKLQSLFDSPDFSKITGKP
IKLTQVEHRAGFEWNEDGAGTTPSPGLQPAHLTFPLDYHLNQPFIFVLRD
TDTGALLFIGKILDPRGP

(SEQ ID NO:19)

DNA Encoding Human PEDF
ATGCAGGCCCTGGTGCTACTCCTCTGCATTGGAGCCCTCCTCGGGCACAG
CAGCTGCCAGAACCCTGCCAGCCCCCCGGAGGAGGGCTCCCCAGACCCCG
ACAGCACAGGGGCGCTGGTGGAGGAGGAGGATCCTTTCTTCAAAGTCCCC
GTGAACAAGCTGGCAGCGGCTGTCTCCAACTTCGGCTATGACCTGTACCG
GGTGCGATCCAGCATGAGCCCCACGACCAACGTGCTCCTGTCTCCTCTCA
GTGTGGCCACGGCCCTCTCGGCCCTCTCGCTGGGAGCGGAGCAGCGAACA
GAATCCATCATTCACCGGGCTCTCTACTATGACTTGATCAGCAGCCCAGA
CATCCATGGTACCTATAAGGAGCTCCTTGACACGGTCACTGCCCCCCAGA
AGAACCTCAAGAGTGCCTCCCGGATCGTCTTTGAGAAGAAGCTRCGCATA
AAATCCAGCTTTGTGGCACCTCTGGAAAAGTCATATGGGACCAGGCCCAG
AGTCCTGACGGGCAACCCTCGCTTGGACCTGCAAGAGATCAACAACTGGG
TGCAGGCGCAGATGAAAGGGAAGCTCGCCAGGTCCACAAAGGAAATTCCC
GATGAGATCAGCATTCTCCTTCTCGGTGTGGCGCACTTCAAGGGGCAGTG
GGTAACAAAGTTTGACTCCAGAAAGACTTCCCTCGAGGATTTCTACTTGG
ATGAAGAGAGGACCGTGAGGGTCCCCATGATGTCGGACCCTAAGGCTGTT
TTACGCTATGGCTTGGATTCAGATCTCAGCTGCAAGATTGCCCAGCTGCC
CTTGACCGGAAGCATGAGTATCATCTTCTTCCTGCCCCTGAAAGTGACCC
AGAATTTGACCTTGATAGAGGAGAGCCTCACCTCCGAGTTCATTCATGAC
ATAGACCGAGAACTGAAGACCGTGCAGGCGGTCCTCACTGTCCCCAAGCT
GAAGCTGAGTTACGAAGGCGAAGTCACCAAGTCCCTGCAGGAGATGAAGC
TGCAATCCTTGTTTGATTCACCAGACTTTAGCAAGATCACAGGCAAACCC
ATCAAGCTGACTCAGGTGGAACACCGGGCTGGCTTTGAGTGGAACGAGGA
TGGGGCGGGAACCACCCCCAGCCCAGGGCTGCAGCCTGCCCACCTCACCT
TCCCGCTGGACTATCACCTTAACCAGCCTTTCATCTTCGTACTGAGGGAC
ACAGACACAGGGGCCCTTCTCTTCATTGGCAAGATTCTGGACCCCAGGGG
CCCCTAA

From PID Accession No. g189778

### 6.1.2 Bovine PEDF

(SEQ ID NO:2)

Bovine PEDF Polypeptide
MQALVLLLWTGALLGFGRCQNAGQEAGSLTPESTGAPVEEEDPFFKVPVN
KLAAAVSNFGYDLYRVRSGESPTANVLLSPLSVATALSASSLGAEQRTES
NIHRALYYDLISNPDIHGTYKDLLASVTAPQKNLKSASRIIFERKLRIKA
SFTPPLEKSYGTRPRILTGNSRVDLQEINNWVQAQMKGKVARSTREMPSE
ISIFLLGVAYFKGQWVTKFDSRKTSLEDFYLDEERTVKVPMMSDPQAVLR
YGLDSDLNCKIAQLPLTGSTSIIFFLPQKVTQNLTLIEESLTSEFTHDID
RELKTVQAVLTIPKLKLSYEGELTKSVQELKLQSLFDAPDFSKITGKPTK
LTQVEHRVGFEWNEDGAGTNSSPGVQPARLTFPLDYHLNQPFTFVLRDTD
TGALLFIGKILDPRGT

(SEQ ID NO:20)

DNA Encoding Bovine PEDF
ATGCAGGCCCTCGTGCTACTCCTCTGGACTGGAGCCCTGCTTGGGTTTGG
CCGCTGCCAGAACGCCGGCCAGGAGGCGGGCTCTCTGACCCCTGAGAGCA
CGGGGGCACCAGTGGAGGAAGAGGATCCCTTCTTCAAGGTCCCTGTGAAC
AAGCTGGCGGCAGCGGTCTCCAACTTCGGCTACGACCTGTACCGCGTGAG
ATCCGGTGAGAGCCCCACCGCCAATGTGCTGCTGTCTCCGCTCAGCGTGG
CCACGGCGCTCTCTGCCCTGTCGCTGGGTGCGGAACAGCGGACAGAATCC
AACATTCACCGGGCTCTGTACTACGACCTGATCAGTAACCCAGACATCCA
CGGCACCTACAAGGACCTCCTTGCCTCCGTCACCGCCCCCCAGAAGAACC
TTAAGAGTGCTTCCCGGATTATCTTTGAGAGGAAGCTGCGGATAAAAGCC
AGCTTCATCCCACCCCTGGAGAAGTCATATGGGACCAGGCCCAGAATCCT
GACCGGCAACTCTCGAGTAGACCTTCAGGAGATTAACAACTGGGTGCAGG
CCCAGATGAAAGGGAAAGTCGCTAGGTCCACGAGGGAGATGCCCAGTGAG
ATCAGCATTTTCCTCCTGGGCGTGGCTTACTTCAAGGGGCAGTGGGTAAC
AAAGTTTGACTCCAGAAAAACTTCCCTGGAGGATTTCTACTTGGATGAGG
AGAGGACCGTGAAAGTCCCCATGATGTCAGACCCTCAGGCCGTTTTACGG
TACGGCTTGGATTCTGATCTCAACTGCAAGATCGCCCAGCTGCCCTTGAC
CGGGAGCACAAGTATCATCTTCTTCCTGCCTCAGAAAGTGACCCAGAACT
TGACCTTGATAGAAGAGAGCCTCACCTCTGAGTTCATTCATGACATAGAC
CGAGAACTGAAGACTGTTCAGGCGGTCCTGACCATTCCCAAGCTGAAGCT
GAGTTATGAAGGCGAACTCACGAAGTCCGTGCAGGAGCTGAAGCTGCAAT
CCCTGTTTGATGCACCAGACTTTAGCAAGATCACAGGCAAACCTATCAAA
CTTACTCAAGTGGAACATCGCGTCGGATTTGAGTGGAATGAGGATGGGGC
GGGTACTAACTCCAGCCCAGGGGTCCAGCCTGCCCGCCTCACCTTCCCTC
TGGACTATCACCTTAACCAACCTTTCATCTTTGTACTGAGGGACACAGAC
ACAGGGGCCCTTCTCTTCATAGGCAAAATTCTGGACCCCAGGGGCACTTA
G

From PID Accession No. g2961474

### 6.1.3 Murine PEDF

(SEQ ID NO:3)

Murine PEDF Polypeptide
MQALVLLLWTGALLGHGSSQNVPSSSEGSPVPDSTGEPVEEEDPFFKVPV
NKLAAAVSNFGYDLYRLRSSASPTGNVLLSPLSVATALSALSLGAEHRTE
SVTHRALYYDLITNPDIHSTYKELLASVTAPEKNLKSASRIVFERKLRVK
SSFVAPLEKSYGTRPRILTGNPRVDLQEINNWVQAQMKGKIARSTREMPS
ALSILLLGVAYFKGQWVTKFDSRKTTLQDFHLDEDRTVRVPMMSDPKAIL
RYGLDSDLNCKIAQLPLTGSMSIIFFLPLAVTQNLTMIEESLTSEFIHDI
DRELKTIQAVLTVPKLKLSFEGELTKSLQDMKLQSLFESPDFSKITGKPV
KLTQVEHRAAFEWNEEGAGSSPSPGLQPVRLTFPLDYHLNQPFLFVLRDT
DTGALLFIGRILDPSST

(SEQ ID NO:21)

DNA Encoding Murine PEDF
ATGCAGGCCCTGGTGCTACTCCTCTGGACTGGAGCCCTGCTCGGGCACGG
CAGCAGCCAGAACGTCCCCAGCAGCTCTGAGGGCTCCCCAGTCCCGGACA
GCACGGGCGAGCCGTGGAGGAGGAGGACCCCTTCTTCAAGGTCCCTGTGA
ACAAGCTGGCAGCAGCTGTCTCCAACTTCGGCTACGATCTGTACCGCCTG
AGATCCAGTGCCAGCCCAACGGGCAACGTCCTGCTGTCTCCACTCAGCGT
GGCCACGGCCCTCTCTGCCCTTTCTCTGGGAGCTGAACATCGAACAGAGT
CTGTCATTCACCGGGCTCTCTACTACGACCTGATCACCAACCCTGACATC
CACAGCACCTACAAGGACTCCTTGCCTCTGTTACTGCCCCTGAGAAGAAC
CTCAAGAGTGCTTCCAGAATTGTGTTTGAGAGGAAACTTCGAGTCAAATC
CAGCTTTGTTGCCCCTCTGGAGAAGTCCTATGGGACCAGGCCCCGGATCC
TCACGGGCAACCCTCGAGTAGACCTTCAGGAGATTAACAACTGGGTGCAG
GCCCAGATGAAAGGGAAGATTGCCCGGTCCACGAGGGAAATGCCCAGTGC
CCTCAGCATCCTTCTCCTTGGGTGGCTTACTTCAAGGGGCAGTGGGTAAC
CAAGTTTGACTCGAGAAAGACGACCCTCCAGGATTTTCATTTGGACGAGG
ACAGGACCGTGAGAGTCCCCATGATGTCAGATCCTAAGGCCATCTTACGA
TACGGCTTGGACTCTGATCTCAACTGCAAGATTGCCCAGCTGCCCTTGAC
AGGAAGTATGAGCATCATCTTCTTCCTGCCCCTGACCGTGACCCAGAACT
TGACCATGATAGAAGAGAGCCTCACCCTGAGTTCATTCATGACATCGACC
GAGAACTGAAGACTATCCAAGCTGTGCTGACTGTCCCCAAGCTGAAGCTG
AGCTTCGAAGGCGAACTTACCAAGTCTCTGCAGGACATGAAGCTACAGTC
GTTGTTTGAATCACCCGACTTCAGCAAGATTACTGGCAAACCCGTGAAGC
TCACCCAAGTGGAACACAGGGCTGCTTTCGAGTGGAATGAAGAGGGGGCA
GGAAGCAGCCCCAGCCCAGGCCTCCAGCCCGTCCGCCTCACCTTCCCGCT
AGACTATCACCTTAACCAACCTTTCCTCTTTGTTCTGAGGGACACGGACA
CGGGGGCCCTCCTCTTCATAGGCAGAATCCTGGACCCCAGTAGCACTTAA

From PID Accession No. g1747298

### 6.2 Human Angiostatin Polypeptide Sequence

(SEQ ID NO:4)

Human Angiostatin Polypeptide
MEHKEVVLLLLLFLKSGQGEPLDDYVNTQGASLFSVTKKQLGAGSIEECA
AKCEEDEEFTCRAFQYHSKEQQCVIMAENRKSSIIIRMRDVVLFEKKVYL
SECKTGNGKNYRGTMSKTKNGITCQKWSSTSPHRPRFSPATHPSEGLEEN
YCRNPDNDPQGPWCYTTDPEKRYDYCDILECEEECMHCSGENYDGKISKT
MSGLECQAWDSQSPIAHGYIPSKFPNKNLKKNYCRNPDRELRPWCFTTDP
NKRWELCDIPRCTTPPPSSGPTYQCLKGTGENYRGNVAVTVSGHTCQHWS
AQTPHTHNRTPENFPCKNLDENYCRNPDGKPAPWCHTTNSQVRWEYCKIP
SCDSSPVSTEQLAPTAPPELTPVVQDCYHGDGQSYRGTSSTTTTGKKCQS
WSSMTPHRHQKTPENYPNAGLTMNYCRNPDADKGPWCFTTDPSVRWEYCN
LKKCSGTEASVVAPPPVVLLPDVETPSEED

From PID Accession No. G130316

### 6.3 Endostatin Polypeptide and Polynucleotide Sequences

### 6.3.1 Human Endostatin

(SEQ ID NO:5)

Human Endostatin Polypeptide
MHSHRDFQPVLHLVALNSPLSGGMRGIRGADFQCFQQARAVGLAGTFRAF
LSSRLQDLYSIVRRADRAAVPIVNLKDELLFPSWEALFSGSEGPLKPGAR
IFSFNGKDVLTHPTWPQKSVWHGSDPNGRRLTESYCETWRTEAPSATGQA
YSLLGGRLLGQSAASCHHAYIVLCIENSFMTASK

(SEQ ID NO:22)

DNA Encoding Human Endostatin
ATGCACAGCCACCGCGACTTCCAGCCGGTGCTCCACCTGGTTGCGCTCAA
CAGCCCCCTGTCAGGCGGCATGCGGGGCATCCGCGGGGCCGACTTCCAGT
GCTTCCAGCAGGCGCGGGCCGTGGGGCTGGCGGGCACCTTCCGCGCCTTC
CTGTCCTCGCGCCTGCAGGACCTGTACAGCATCGTGCGCCGTGCCGACCG
CGCAGCCGTGCCCATCGTCAACCTCAAGGACGAGCTGCTGTTTCCCAGCT
GGGAGGCTCTGTTCTCAGGCTCTGAGGGTCCGCTGAAGCCCGGGGCACGC
ATCTTCTCCTTTAACGGCAAGGACGTCCTGACCCACCCCACCTGGCCCCA
GAAGAGCGTGTGGCATGGCTCGGACCCCAACGGGCGCAGGCTGACCGAGA
GCTACTGTGAGACGTGGCGGACGGAGGCTCCCTCGGCCACGGGCCAGGCC
TACTCGCTGCTGGGGGGCAGGCTCCTGGGGCAGAGTGCCGCGAGCTGCCA
TCACGCCTACATCGTGCTATGCATTGAGAACAGCTTCATGACTGCCTCCA
AGTAG

From GenBank Accession No. AF184060 (Zhi-Hong et al., 1999)

### 6.3.2 Murine Endostatin

(SEQ ID NO:6)

Murine Endostatin Polypeptide
HTHQDFQPVLHLVALNTPLSGGMRGIRGADFQCFQQARAVGLSGTFRAFL
SSRLQDLYSIVRRADRGSVPIVNLKDEVLSPSWDSLFSGSQGQLQPGARI
FSFDGRDVLRHPAWPQKSVWHGSDPSGRRLMESYCETWRTETTGATGQAS
SLLSGRLLEQKAASCHNSYIVLCIENSFMTSFSK

(SEQ ID NO:23)

DNA Encoding Murine Endostatin
CATACTCATCAGGACTTTCAGCCAGTGCTCCACCTGGTGGCACTGAACAC
CCCCCTGTCTGGAGGCATGCGTGGTATCCGTGGAGCAGATTTCCAGTGCT
TCCAGCAAGCCCGAGCCGTGGGGCTGTCGGGCACCTTCCGGGCTTTCCTG
TCCTCTAGGCTGCAGGATCTCTATAGCATCGTGCGCCGTGCTGACCGGGG
GTCTGTGCCCATCGTCAACCTGAAGGACGAGGTGCTATCTCCCAGCTGGG
ACTCCCTGTTTTCTGGCTCCCAGGGTCAACTGCAACCCGGGGCCCGCATC
TTTTCTTTTGACGGCAGAGATGTCCTGAGACACCCAGCCTGGCCGCAGAA
GAGCGTATGGCACGGCTCGGACCCCAGTGGGCGGAGGCTGATGGAGAGTT
ACTGTGAGACATGGCGAACTGAAACTACTGGGGCTACAGGTCAGGCCTCC
TCCCTGCTGTCAGGCAGGCTCCTGGAACAGAAAGCTGCGAGCTGCCACAA
CAGCTACATCGTCCTGTGCATTGAGAATAGCTTCATGACCTCTTTCTCCA
AA

From GenBank Accession No. AF257775

### 6.4 TIMP3 Polypeptide and Polynucleotide Sequences

### 6.4.1 Bovine TIMP3

(SEQ ID NO:7)

Bovine TIMP3 Polypeptide
MTPWLGLVVLLGSWSLGDWGAEACTCSPSHPQDAFCNSDIVIRAKVVGKK
LLKEGPFGTMVYTIKQMKMYRGFTKMPHVQYIHTEASESLCGLKLEVNKY
QYLLTGRVYDGKMYTGLCNFVERWDQLTLSQRKGLNYRYHLGCNCKIKSC
YYLPCFVTSKNECLWTDMFSNFGYPGYQSKHYACIRQKGGYCSWYRGWAP
PDKSIINATDP

(SEQ ID NO:24)

DNA Encoding Bovine TIMP3
TGCACATGCTCGCCTAGCCACCCCCAGGACGCGTTCTGCAACTCAGACAT
CGTGATCCGAGCCAAGGTGGTAGGGAAGAAACTGCTGAAGGAGGGGCCCT
TTGGCACGATGGTCTACACCATCAAGCAGATGAAGATGTACCGAGGATTC
ACCAAGATGCCCCATGTGCAGTACATCCACACAGAAGCTTCTGAAAGTCT
CTGTGGCCTTAAGCTTGAGGTCAACAAGTACCAGTACCTGCTGACAGGCC
GAGTCTATGATGGCAAGATGTACACAGGACTGTGTAACTTTGTAGAGAGG
TGGGACCAGCTCACCCTCTCCCAGCGCAAGGGGCTGAACTATCGATATCA
CCTGGGCTGTAACTGCAAGATCAAATCCTGCTACTACCTGCCTTGCTTTG
TAACCTCCAAGAACGAGTGTCTCTGGACCGACATGTTCTCCAATTTCGGC
TACCCTGGCTACCAGTCCAAACACTACGCTTGCATCCGGCAGAAGGGTGG
CTACTGTAGCTGGTACCGAGGATGGGCACCCCCGGACAAAAGCATCATCA
ATGCCACAGACCCCTGA

From GenBank Accession No. NM_{—}174473 (Criado et al., “Primary structure of an agonist binding subunit of the nicotinic acetylcholine receptor from bovine adrenal chromaffin cells,” Neurochem. Res., 17(3):281-287, 1992).

### 6.4.2 Rat TIMP3

(SEQ ID NO:8)

Rat TIMP3 Polypeptide
MTPWLGLVVLLSCWSLGHWGTEACTCSPSHPQDAFCNSDIVIPAKVVGKK
LVKEGPFGTLVYTIKQMKMYRGFSKMPHVQYIHTEASESLCGLKLEVNKY
QYLLTGRVYEGKMYTGLCNFVERWDHLTLSQRKGLNYRYHLGCNCKIKSC
YYLPCFVTSKKECLWTDMLSNFGYPGYQSKHYACIRQKGGYCSWYRGWAP
PDKSISNATDP

(SEQ ID NO:25)

DNA Encoding Rat TIMP3
ATGACTCCCTGGCTTGGGCTTGTCGTGCTCCTGAGCTGCTGGAGCCTTGG
GCACTGGGGAACGGAAGCGTGCACATGCTCGCCCAGCCATCCCCAGGATG
CCTTCTGCAACTCCGACATCGTGATCCGGGCCAAAGTGGTGGGAAAGAAG
CTGGTGAAGGAAGGGCCCTTTGGCACTCTGGTCTACACTATTAAGCAAAT
GAAGATGTACCGAGGATTCAGTAAGATGCCCCATGTGCAGTACATTCACA
CGGAAGCCTCTGAAAGTCTCTGTGGCCTTAAGCTAGAAGTCAACAAATAC
CAGTACCTGCTGACAGGGCGCGTGTATGAAGGCAAGATGTACACAGGGCT
GTGCAACTTTGTGGAGAGGTGGGACCACCTCACACTGTCCCAGCGCAAGG
GCCTCAATTACCGCTACCACCTGGGTTGCAATTGCAAGATCAAGTCCTGC
TACTACTTGCCTTGCTTTGTGACCTCCAAGAAAGAATGTCTCTGGACCGA
CATGCTCTCCAATTTCGGGTACCCTGGCTATCAGTCCAAACACTACGCCT
GCATCCGGCAGAAGGGTGGCTACTGCAGCTGGTACCGAGGATGGGCCCCC
CCAGACAAGAGCATCAGCAATGCCACAGACCCCTGA

From GenBank Accession No. NM_{—}012886 (Tanaka et al., “Clinical consideration with special reference to autopsy cases of malignant tumor in the oral cavity, treated with Bleomycin,” Hiroshima Daigaku Shigaku Zasshi, 8(2):168-175, 1975).

### 6.5 sFLT Polypeptide and Polynucleotide Sequences

### 6.5.1 Human sFLT

(SEQ ID NO:9)

Human SFLT1 Polypeptide
MVSYWDTGVLLCALLSCLLLTGSSSGSKLKDPELSLKGTQHIMQAGQTLH
LQCRGEAAHKWSLPEMVSKESERLSITKSACGRNGKQFCSTLTLNTAQAN
HTGFYSCKYLAVPTSKKKETESAIYIFISDTGRPFVEMYSEIPEIIHMTE
GRELVIPCRVTSPNITVTLKKFPLDTLIPDGKRIIWDSRKGFIISNATYK
EIGLLTCEATVNGHLYKTNYLTHRQTNTIIDVQISTPRPVKLLRGHTLVL
NCTATTPLNTRVQMTWSYPDEKNKRASVRRRIDQSNSHANIFYSVLTIDK
MQNKDKGLYTCRVRSGPSFKSVNTSVHIYDKAFITVKHRKQQVLETVAGK
RSYRLSMKVKAFPSPEVVWLKDGLPATEKSARYLTRGYSLIIKDVTEEDA
GNYTILLSIKQSNVFKNLTATLIVNVKPQIYEKAVSSFPDPALYPLGSRQ
ILTCTAYGIPQPTIKWFWHPCNHNHSEARCDFCSNNEESFILDADSNMGN
RIESITQRMAIIEGKNKMASTLVVADSRISGIYICIASNKVGTVGRNISF
YITDVPNGFHVNLEKMPTEGEDLKLSCTVNKFLYRDVTWILLRTVNNRTM
HYSISKQKMAITKEHSITLNLTIMNVSLQDSGTYACRARNVYTGEEILQK
KEITIRGEHCNKKAVFSRISKFKSTRNDCTTQSNVKH

(SEQ ID NO:26)

DNA Encoding Human SFLT1
ATGGTCAGCTACTGGGACACCGGGGTCCTGCTGTGCGCGCTGCTCAGCTG
TCTGCTTCTCACAGGATCTAGTTCAGGTTCAAAATTAAAAGATCCTGAAC
TGAGTTTAAAAGGCACCCAGCACATCATGCAAGCAGGCCAGACACTGCAT
CTCCAATGCAGGGGGGAAGCAGCCCATAAATGGTCTTTGCCTGAAATGGT
GAGTAAGGAAAGCGAAAGGCTGAGCATAACTAAATCTGCCTGTGGAAGAA
ATGGCAAACAATTCTGCAGTACTTTAACCTTGAACACAGCTCAAGCAAAC
CACACTGGCTTCTACAGCTGCAAATATCTAGCTGTACCTACTTCAAAGAA
GAAGGAAACAGAATCTGCAATCTATATATTTATTAGTGATACAGGTAGAC
CTTTCGTAGAGATGTACAGTGAAATCCCCGAAATTATACACATGACTGAA
GGAAGGGAGCTCGTCATTCCCTGCCGGGTTACGTCACCTAACATCACTGT
TACTTTAAAAAAGTTTCCACTTGACACTTTGATCCCTGATGGAAAACGCA
TAATCTGGGACAGTAGAAAGGGCTTCATCATATCAAATGCAACGTACAAA
GAAATAGGGCTTCTGACCTGTGAAGCAACAGTCAATGGGCATTTGTATAA
GACAAACTATCTCACACATCGACAAACCAATACAATCATAGATGTCCAAA
TAAGCACACCACGCCCAGTCAAATTACTTAGAGGCCATACTCTTGTCCTC
AATTGTACTGCTACCACTCCCTTGAACACGAGAGTTCAAATGACCTGGAG
TTACCCTGATGAAAAAAATAAGAGAGCTTCCGTAAGGCGACGAATTGACC
AAAGCAATTCCCATGCCAACATATTCTACAGTGTTCTTACTATTGACAAA
ATGCAGAACAAAGACAAAGGACTTTATACTTGTCGTGTAAGGAGTGGACC
ATCATTCAAATCTGTTAACACCTCAGTGCATATATATGATAAAGCATTCA
TCACTGTGAAACATCGAAAACAGCAGGTGCTTGAAACCGTAGCTGGCAAG
CGGTCTTACCGGCTCTCTATGAAAGTGAAGGCATTTCCCTCGCCGGAAGT
TGTATGGTTAAAAGATGGGTTACCTGCGACTGAGAAATCTGCTCGCTATT
TGACTCGTGGCTACTCGTTAATTATCAAGGACGTAACTGAAGAGGATGCA
GGGAATTATACAATCTTGCTGAGCATAAAACAGTCAAATGTGTTTAAAAA
CCTCACTGCCACTCTAATTGTCAATGTGAAACCCCAGATTTACGAAAAGG
CCGTGTCATCGTTTCCAGACCCGGCTCTCTACCCACTGGGCAGCAGACAA
ATCCTGACTTGTACCGCATATGGTATCCCTCAACCTACAATCAAGTGGTT
CTGGCACCCCTGTAACCATAATCATTCCGAAGCAAGGTGTGACTTTTGTT
CCAATAATGAAGAGTCCTTTATCCTGGATGCTGACAGCAACATGGGAAAC
AGAATTGAGAGCATCACTCAGCGCATGGCAATAATAGAAGGAAAGAATAA
GATGGCTAGCACCTTGGTTGTGGCTGACTCTAGAATTTCTGGAATCTACA
TTTGCATAGCTTCCAATAAAGTTGGGACTGTGGGAAGAAACATAAGCTTT
TATATCACAGATGTGCCAAATGGGTTTCATGTTAACTTGGAAAAAATGCC
GACGGAAGGAGAGGACCTGAAACTGTCTTGCACAGTTAACAAGTTCTTAT
ACAGAGACGTTACTTGGATTTTACTGCGGACAGTTAATAACAGAACAATG
CACTACAGTATTAGCAAGCAAAAAATGGCCATCACTAAGGAGCACTCCAT
CACTCTTAATCTTACCATCATGAATGTTTCCCTGCAAGATTCAGGCACCT
ATGCCTGCAGAGCCAGGAATGTATACACAGGGGAAGAAATCCTCCAGAAG
AAAGAAATTACAATCAGAGGTGAGCACTGCAACAAAAAGGCTGTTTTCTC
TCGGATCTCCAAATTTAAAAGCACAAGGAATGATTGTACCACACAAAGTA
ATGTAAAACATTAA

From GenBank Accession No. U01134 (Kendall and Thomas “Inhibition of vascular endothelial cell growth factor activity by an endogenously encoded soluble receptor,” Proc. Natl. Acad. Sci. USA, 90(22):10705-10709, 1993).

### 6.5.2 Murine sFLT

(SEQ ID NO:10)

Murine SFLT1 Polypeptide
MVSCWDTAVLPYALLGCLLLTGYGSGSKLKVPELSLKGTQHVMQAGQTLF
LKCRGEAAHSWSLPTTVSQEDKRLSITPPSACGRDNRQFCSTLTLDTAQA
NHTGLYTCRYLPTSTSKKKKAESSIYIFVSDAGSPFIEMHTDIPKLVHMT
EGRQLIIPCRVTSPNVTVTLKKFPFDTLTPDGQRITWDSRRGFIIANATY
KEIGLLNCEATVNGHLYQTNYLTHRQTNTILDVQIRPPSPVRLLHGQTLV
LNCTATTELNTRVQMSWNYPGKATKRASIRQRIDRSHSHNNVFHSVLKIN
NVESRDKGLYTCRVKSGSSFQSFNTSVHVYEKGFISVKHRKQPVQETTAG
RRSYRLSMKVKAFPSPEIVWLKDGSPATLKSARYLVHGYSLIIKDVTTED
AGDYTILLGIKQSRLFKNLTATLIVNVKPQIYEKSVSSLPSPPLYPLGSR
QVLTCTVYGIPRPTITWLWHPCHHNHSKERYDFCTENEESFILDPSSNLG
NRIESISQRMTVIEGTNKTVSTLVVADSQTPGIYSCRAFNKIGTVERNIK
FYVTDVPNGFHVSLEKMPAEGEDLKLSCVVNKFLYRDITWILLRTVNNRT
MHHSISKQKMATTQDYSITLNLVIKNVSLEDSGTYACRARNIYTGEDILR
KTEVLVRGEHCGKKAIFSRISKFKSRRNDCTTQSHVKH

(SEQ ID NO:27)

DNA Encoding Murine SFLT1
ATGGTCAGCTGCTGGGACACCGCGGTCTTGCCTTACGCGCTGCTCGGGTG
TCTGCTTCTCACAGGATATGGCTCAGGGTCGAAGTTAAAAGTGCCTGAAC
TGAGTTTAAAAGGCACCCAGCATGTCATGCAAGCAGGCCAGACTCTCTTT
CTCAAGTGCAGAGGGGAGGCAGCCCACTCATGGTCTCTGCCCACGACCGT
GAGCCAGGAGGACAAAAGGCTGAGCATCACTCCCCCATCGGCCTGTGGGA
GGGATAACAGGCAATTCTGCAGCACCTTGACCTTGGACACGGCGCAGGCC
AACCACACGGGCCTCTACACCTGTAGATACCTCCCTACATCTACTTCGAA
GAAAAAGAAAGCGGAATCTTCAATCTACATATTTGTTAGTGATGCAGGGA
GTCCTTTCATAGAGATGCACACTGACATACCCAAACTTGTGCACATGACG
GAAGGAAGACAGCTCATCATCCCCTGCCGGGTGACGTCACCCAACGTCAC
AGTCACCCTAAAAAAGTTTCCATTTGATACTCTTACCCCTGATGGGCAAA
GAATAACATGGGACAGTAGGAGAGGCTTTATAATAGCAAATGCAACGTAC
AAAGAGATAGGACTGCTGAACTGCGAAGCCACCGTCAACGGGCACCTGTA
CCAGACAAACTATCTGACCCATCGGCAGACCAATACAATCCTAGATGTCC
AAATACGCCCGCCGAGCCCAGTGAGACTGCTCCACGGGCAGACTCTTGTC
CTCAACTGCACCGCCACCACGGAGCTCAATACGAGGGTGCAAATGAGCTG
GAATTACCCTGGTAAAGCAACTAAGAGAGCATCTATAAGGCAGCGGATTG
ACCGGAGCCATTCCCACAACAATGTGTTCCACAGTGTTCTTAAGATCAAC
AATGTGGAGAGCCGAGACAAGGGGCTCTACACCTGTCGCGTGAAGAGTGG
GTCCTCGTTCCAGTCTTTCAACACCTCCGTGCATGTGTATGAAAAAGGAT
TCATCAGTGTGAAACATCGGAAGCAGCCGGTGCAGGAAACCACAGCAGGA
AGACGGTCCTATCGGCTGTCCATGAAAGTGAAGGCCTTCCCCTCCCCAGA
AATCGTATGGTTAAAAGATGGCTCGCCTGCAACATTGAAGTCTGCTCGCT
ATTTGGTACATGGCTACTCATTAATTATCAAAGATGTGACAACCGAGGAT
GCAGGGGACTATACGATCTTGCTGGGCATAAAGCAGTCAAGGCTATTTAA
AAACCTCACTGCCACTCTCATTGTAAACGTGAAACCTCAGATCTACGAAA
AGTCCGTGTCCTCGCTTCCAAGCCCACCTCTCTATCCGCTGGGCAGCAGA
CAAGTCCTCACTTGCACCGTGTATGGCATCCCTCGGCCAACAATCACGTG
GCTCTGGCACCCCTGTCACCACAATCACTCCAAAGAAAGGTATGACTTCT
GCACTGAGAATGAAGAATCCTTTATCCTGGATCCCAGCAGCAACTTAGGA
AACAGAATTGAGAGCATCTCTCAGCGCATGACGGTCATAGAAGGAACAAA
TAAGACGGTTAGCACATTGGTGGTGGCTGACTCTCAGACCCCTGGAATCT
ACAGCTGCCGGGCCTTCAATAAAATAGGGACTGTGGAAAGAAACATAAAA
TTTTACGTCACAGATGTGCCGAATGGCTTTCACGTTTCCTTGGAAAAGAT
GCCAGCCGAAGGAGAGGACCTGAAACTGTCCTGTGTGGTCAATAAATTCC
TGTACAGAGACATTACCTGGATTCTGCTACGGACAGTTAACAACAGAACC
ATGCACCATAGTATCAGCAAGCAAAAAATGGCCACCACTCAAGATTACTC
CATCACTCTGAACCTTGTCATCAAGAACGTGTCTCTAGAAGACTCGGGCA
CCTATGCGTGCAGAGCCAGGAACATATACACAGGGGAAGACATCCTTCGG
AAGACAGAAGTTCTCGTTAGAGGTGAGCACTGCGGCAAAAAGGCCATTTT
CTCTCGGATCTCCAAATTTAAAAGCAGGAGGAATGATTGTACCACACAAA
GTCATGTCAAACATTAA

From GenBank Accession Number D88690 (Finnerty et al., “Molecular cloning of murine FLT and FLT4,” Oncogene, 8(8):2293-2298, 1993).

### 6.6 Biologically-Active VEGF Peptides and Polynucleotide Sequences

VEGF Exon 6 Peptide (AA121-132 of VEGF)

(SEQ ID NO:11)

KGKGQKRKRKKS
DNA Encoding VEGF Exon 6 Peptide

(SEQ ID NO:28)

AAGGGAAAGGGGCAAAAACGAAAGCGCAAGAAATCC
VEGF Exon 7 Peptide
(AA22-44 of Exon7 and first Cys of Exon8)

(SEQ ID NO:12)

CSCKNTDSRCKARQLELNERTCRC
DNA Sequence Encoding VEGF Exon 7 Peptide

(SEQ ID NO:29)

TGTTCCTGCAAAAACACAGACTCGCGTTGCAAGGCGAGGCAGCTTGAGTT
AAACGAACGTACTTGCAGATGT

### 6.7 Tmrombospondin Polypeptide and Polynucleotide Sequences

(SEQ ID NO:13)

Human Thrombospondin Polypeptide
MRKGLRATAARCGLGLGYLLQMLVLPALALLSASGTGSAAQDDDFFHELP
ETFPSDPPEPLPHFLIEPEEAYIVKNKPVNLYCKASPATQIYFKCNSEWV
HQKDHIVDERVDETSGLIVREVSIEISRQQVEELFGPEDYWCQCVAWSSA
GTTKSRKAYVRIAYLRKTFEQEPLGKEVSLEQEVLLQCRPPEGIPVAEVE
WLKNEDIIDPVEDRNFYITIDHNLIIKQARLSDTANYTCVAKNIVAKRKS
TTATVIVYVNGGWSTWTEWSVCNSRCGRGYQKRTRTCTNPAPLNGGAFCE
GQSVQKIACTTLCPVDGRWTPWSKWSTCGTECTHWRRRECTAPAPKNGGK
DCDGLVLQSKNCTDGLCMQTAPDSDDVALYVGIVIAVIVCLAISVVVALF
VYRKNHRDFESDIIDSSALNGGFQPVNIKAARQDLLAVPPDLTSAAAMYR
GPVYALHDVSDKIPMTNSPILDPLPNLKIKVYNTSGAVTPQDDLSEFTSK
LSPQMTQSLLENEALSLKNQSLARQTDPSCTAFGSFNSLGGHLIVPNSGV
SLLIPAGAIPQGRVYEMYVTVHRKETMRPPMDDSQTLLTPVVSCGPPGAL
LTRPVVLTMHHCADPNTEDWKILLKNQAAQGQWEDVVVVGEENFTTPCYI
KLDAEACHILTENLSTYALVGHSTTKAAAKRLKLAIFGPLCCSSLEYSIR
VYCLDDTQDALKEILHLERQTGGQLLEEPKALHFKGSTHNLRLSIHDIAH
SLWKSKLLAKYQEIPFYHVWSGSQRNLHCTFTLERFSLNTVELVCKLCVR
QVEGEGQIFQLNCTVSEEPTGIDLPLLDPANTITTVTGPSAFSIPLPIRQ
KLCSSLDAPQTRGHDWRMLAHKLNLDRYLNYFATKSSPTGVILDLWEAQN
FPDGNLSMLAAVLEEMGRHETVVSLAAEGQY

(SEQ ID NO:30)

MDNA Encoding Human Thrombospondin
ATGAGGAAAGGTCTGCGGGCGACAGCGGCCCGCTGCGGACTGGGACTGGG
ATACTTGCTGCAAATGCTCGTGCTACCTGCCCTGGCCCTGCTCAGCGCCA
GCGGCACTGGCTCCGCCGCCCAAGATGATGACTTTTTTCATGAACTCCCA
GAAACTTTTCCTTCTGATCCACCTGAGCCTCTGCCACATTTCCTTATTGA
GCCTGAAGAAGCTTATATTGTGAAGAATAAGCCTGTGAACCTGTACTGTA
AAGCAAGCCCTGCCACCCAGATCTATTTCAAGTGTAATAGTGAATGGGTT
CATCAGAAGGACCACATAGTAGATGAAAGAGTAGATGAAACTTCCGGTCT
CATTGTCCGGGAAGTGAGCATTGAGATTTCGCGCCAGCAAGTGGAAGAAC
TCTTTGGACCTGAAGATTACTGGTGCCAGTGTGTGGCCTGGAGCTCCGCG
GGTACCACAAAGAGCCGGAAGGCGTATGTGCGCATTGCATATCTACGGAA
GACATTTGAGCAGGAACCCCTAGGAAAGGAAGTGTCTTTGGAACAGGAAG
TCTTACTCCAGTGTCGACCACCTGAAGGGATCCCAGTGGCTGAGGTGGAA
TGGTTGAAAAATGAAGACATAATTGATCCCGTTGAAGATCGGAATTTTTA
TATTACTATTGATCACAACCTCATCATAAAGCAGGCCCGACTCTCTGATA
CTGCAAATTACACCTGTGTTGCCAAAAACATTGTTGCCAAGAGGAAAAGT
ACAACTGCCACTGTCATAGTCTATGTCAACGGTGGCTGGTCCACCTGGAC
GGAGTGGTCTGTGTGTAACAGCCGCTGTGGACGAGGGTATCAGAAACGTA
CAAGGACTTGTACCAACCCGGCACCACTCAATGGGGGTGCCTTCTGTGAA
GGGCAGAGTGTGCAGAAAATAGCCTGTACTACGTTATGCCCAGTGGATGG
CAGGTGGACGCCATGGAGCAAGTGGTCTACTTGTGGAACTGAGTGCACCC
ACTGGCGCAGGAGGGAGTGCACGGCGCCAGCCCCCAAGAATGGAGGCAAG
GACTGCGACGGCCTCGTCTTGCAATCCAAGAACTGCACTGATGGGCTTTG
CATGCAGACTGCTCCTGATTCAGATGATGTTGCTCTCTATGTTGGGATTG
TGATAGCAGTGATCGTTTGCCTGGCGATCTCTGTAGTTGTGGCCTTGTTT
GTGTATCGGAAGAATCATCGTGACTTTGAGTCAGATATTATTGACTCTTC
GGCACTCAATGGGGGCTTTCAGCCTGTGAACATCAAGGCAGCAAGACAAG
ATCTGCTGGCTGTACCCCCAGACCTCACGTCAGCTGCAGCCATGTACAGA
GGACCTGTCTATGCCCTGCATGACGTCTCAGACAAAATCCCAATGACCAA
CTCTCCAATTCTGGATCCACTGCCCAACCTGAAAATCAAAGTGTACAACA
CCTCAGGTGCTGTCACCCCCCAAGATGACCTCTCTGAGTTTACGTCCAAG
CTGTCCCCTCAGATGACCCAGTCGTTGTTGGAGAATGAAGCCCTCAGCCT
GAAGAACCAGAGTCTAGCAAGGCAGACTGATCCATCCTGTACCGCATTTG
GCAGCTTCAACTCGCTGGGAGGTCACCTTATTGTTCCCAATTCAGGAGTC
AGCTTGCTGATTCCCGCTGGGGCCATTCCCCAAGGGAGAGTCTACGAAAT
GTATGTGACTGTACACAGGAAAGAAACTATGAGGCCACCCATGGATGACT
CTCAGACACTTTTGACCCCTGTGGTGAGCTGTGGGCCCCCAGGAGCTCTG
CTCACCCGCCCCGTCGTCCTCACTATGCATCACTGCGCAGACCCCAATAC
CGAGGACTGGAAAATACTGCTCAAGAACCAGGCAGCACAGGGACAGTGGG
AGGATGTGGTGGTGGTCGGGGAGGAAAACTTCACCACCCCCTGCTACATT
AAGCTGGATGCAGAGGCCTGCCACATCCTCACAGAGAACCTCAGCACCTA
CGCCCTGGTAGGACATTCCACCACCAAAGCGGCTGCAAAGCGCCTCAAGC
TGGCCATCTTTGGGCCCCTGTGCTGCTCCTCGCTGGAGTACAGCATCCGA
GTCTACTGTCTGGATGACACCCAGGATGCCCTGAAGGAAATTTTACATCT
TGAGAGACAGACGGGAGGACAGCTCCTAGAAGAACCTAAGGCTCTTCATT
TTAAAGGCAGCACCCACAACCTGCGCCTGTCAATTCACGATATCGCCCAT
TCCCTCTGGAAGAGCAAATTGCTGGCTAAATATCAGGAAATTCCATTTTA
CCATGTTTGGAGTGGATCTCAAAGAAACCTGCACTGCACCTTCACTCTGG
AAAGATTTAGCCTGAACACAGTGGAGCTGGTTTGCAAACTCTGTGTGCGG
CAGGTGGAAGGAGAAGGGCAGATCTTCCAGCTCAACTGCACCGTGTCAGA
GGAACCTACTGGCATCGATTTGCCGCTGCTGGATCCTGCGAACACCATCA
CCACGGTCACGGGGCCCAGTGCTTTCAGCATCCCTCTCCCTATCCGGCAG
AAGCTCTGTAGCAGCCTGGATGCCCCCCAGACGAGAGGCCATGACTGGAG
GATGCTGGCCCATAAGCTGAACCTGGACAGGTACTTGAATTACTTTGCCA
CCAAATCCAGCCCAACTGGCGTAATCCTGGATCTTTGGGAAGCACAGAAC
TTCCCAGATGGAAACCTGAGCATGCTGGCAGCTGTCTTGGAAGAAATGGG
AAGACATGAAACGGTGGTGTCCTTAGCAGCAGAAGGGCAGTATTAA

From GenBank Accession Number NM_{—}003728 (Ackerman and Knowles, “Cloning and mapping of the UNC5C gene to human chromosome 4q21-q23,” Genomics, 52(2):205-208, 1998).

### 6.8 Tryptophanyl-tRNA Synthetase Polypeptide and Polynucleotide Sequences

(SEQ ID NO:14)

Human Tryptophanyl-tRNA Synthetase Polypeptide
MPNSEPASLLELFNSIATQGELVRSLKAGNASKDEIDSAVKMLVSLKMSY
KAAAGEDYKADCPPGNPAPTSNHGPDATEAEEDFVDPWTVQTSSAKGIDY
DKLIVRFGSSKIDKELINRIEPATGQRPHHFLRRGIFFSHRDMNQVLDAY
ENKKPFYLYTGRGPSSEAMHVGHLIPFIFTKWLQDVFNVPLVIQMTDDEK
YLWKDLTLDQAYGDAVENAKDIIACGFDINKTFIFSDLDYMGMSSGFYKN
VVKIQKHVTFNQVKGIFGFTDSDCIGKISFPAIQAAPSFSNSFPQIFRDR
TDIQCLIPCAIDQDPYFRNTRDVAPRIGYPKPALLHSTFFPALQGAQTKM
SASDPNSSIFLTDTAKQIKTKVNKHAFSGGRDTIEEHRQFGGNCDVDVSF
MYLTFFLEDDDKLEQIRKDYTSGAMLTGELKKALTEVLQPLIAEHQARRK
EVTDEIVKEFMTPRKLSFDFQ

(SEQ ID NO:31)

DNA Encoding Human Tryptophanyl-tRNA Synthetase
ATGCCCAACAGTGAGCCCGCATCTCTGCTGGAGCTGTTCAACAGCATCGC
CACACAAGGGGAGCTCGTAAGGTCCCTCAAAGCGGGAAATGCGTCAAAGG
ATGAAATTGATTCTGCAGTAAAGATGTTGGTGTCATTAAAAATGAGCTAC
AAAGCTGCCGCGGGGGAGGATTACAAGGCTGACTGTCCTCCAGGGAACCC
AGCACCTACCAGTAATCATGGCCCAGATGCCACAGAAGCTGAAGAGGATT
TTGTGGACCCATGGACAGTACAGACAAGCAGTGCAAAAGGCATAGACTAC
GATAAGCTCATTGTTCGGTTTGGAAGTAGTAAAATTGACAAAGAGCTAAT
AAACCGAATAGAGAGAGCCACCGGCCAAAGACCACACCACTTCCTGCGCA
GAGGCATCTTCTTCTCACACAGAGATATGAATCAGGTTCTTGATGCCTAT
GAAAATAAGAAGCCATTTTATCTGTACACGGGCCGGGGCCCCTCTTCTGA
AGCAATGCATGTAGGTCACCTCATTCCATTTATTTTCACAAAGTGGCTCC
AGGATGTATTTAACGTGCCCTTGGTCATCCAGATGACGGATGACGAGAAG
TATCTGTGGAAGGACCTGACCCTGGACCAGGCCTATGGCGATGCTGTTGA
GAATGCCAAGGACATCATCGCCTGTGGCTTTGACATCAACAAGACTTTCA
TATTCTCTGACCTGGACTACATGGGGATGAGCTCAGGTTTCTACAAAAAT
GTGGTGAAGATTCAAAAGCATGTTACCTTCAACCAAGTGAAAGGCATTTT
CGGCTTCACTGACAGCGACTGCATTGGGAAGATCAGTTTTCCTGCCATCC
AGGCTGCTCCCTCCTTCAGCAACTCATTCCCACAGATCTTCCGAGACAGG
ACGGATATCCAGTGCCTTATCCCATGTGCCATTGACCAGGATCCTTACTT
TAGAATGACAAGGGACGTCGCCCCCAGGATCGGCTATCCTAAACCAGCCC
TGTTGCACTCCACCTTCTTCCCAGCCCTGCAGGGCGCCCAGACCAAAATG
AGTGCCAGCGACCCAAACTCCTCCATCTTCCTCACCGACACGGCCAAGCA
GATCAAAACCAAGGTCAATAAGCATGCGTTTTCTGGAGGGAGAGACACCA
TCGAGGAGCACAGGCAGTTTGGGGGCAACTGTGATCTGGACGTGTCTTTC
ATGTACCTGACCTTCTTCCTCGAGGACGACGACAAGCTCGAGCAGATCAG
GAAGGATTACACCAGCGGAGCCATGCTCACCGGTGAGCTCAAGAAGGCAC
TCATAGAGGTTCTGCAGCCCTTGATCGCAGAGCACCAGGCCCGGCGCAAG
GAGGTCACGGATGAGATAGTGAAAGAGTTCATGACTCCCCGGAAGCTGTC
CTTCGACTTTCAGTAG

From GenBank Accession Number NM_{—}004184 (Fleckner et al., Proc. Natl. Acad. Sci. USA, 88 (24), 11520-11524, 1991).

### 6.9 Tyrosyl-tRNA Synthetase Polypeptide and Polynucleotide Sequences

(SEQ ID NO:15)

Human Tyrosyl-tRNA SynthetasePolypeptide
MGDAPSPEEKLHLITRNLQEVLGEEKLKEILKERELKIYWGTATTGKPHV
AYFVPMSKIADFLKAGCEVTILFADLHAYLDNMKAPWELLELRVSYYENV
IKAMLESIGVPLEKLKFIKGTDYQLSKEYTLDVYRLSSVVTQHDSKKAGA
EVVKQVEHPLLSGLLYPGLQALDEEYLKVDAQFGGIDQRKIFTFAEKYLP
ALGYSKRVHLMNPMVPGLTGSKMSSSEEESKIDLLDRKEDVKKKLKKACE
PGNVENNGVLSFIKHVLFPLKSEFVILRDEKWGGNKTYTAYVDLEKDFAA
EVVHPGDLKNSVEVALNKLLDPTREKFNTPALKKLASAAYPDPSKQKPMA
KGPAKNSEPEEVIPSRLDIRVGKIITVEKHPDADSLYVEKIDVGEAEPRT
VVSGLVQFVPKEELQDRLVVVLCNLKPQKMRGVESQGMLLCASTEGTNRQ
VEPLDPPAGSAPGEHVFVKGYEKGQPDEELKPKKKVFEKLQADFKISEEC
IAQWKQTNFMTKLGSISCKSLKGGNIS

(SEQ ID NO:32)

DNA Encoding Human Tryrosyl-tRNA Synthetase
ATGGGGGACGCTCCCAGCCCTGAAGAGAAACTGCACCTTATCACCCGGAA
CCTGCAGGAGGTTCTGGGGGAAGAGAAGCTGAAGGAGATACTGAAGGAGC
GGGAACTTAAAATTTACTGGGGAACGGCAACCACGGGCAAACCACATGTG
GCTTACTTTGTGCCCATGTCAAAGATTGCAGACTTCTTAAAGGCAGGGTG
TGAGGTAACAATTCTGTTTGCGGACCTCCACGCATACCTGGATAACATGA
AAGCCCCATGGGAACTTCTAGAACTCCGAGTCAGTTACTATGAGAATGTG
ATCAAAGCAATGCTGGAGAGCATTGGTGTGCCCTTGGAGAAGCTCAAGTT
CATCAAAGGCACTGATTACCAGCTCAGCAAAGAGTACACACTAGATGTGT
ACAGACTCTCCTCCGTGGTCACACAGCACGATTCCAAGAAGGCTGGAGCT
GAGGTGGTAAAGCAGGTGGAGCACCCTTTGCTGAGTGGCCTCTTATACCC
CGGACTGCAGGCTTTGGATGAAGAGTATTTAAAAGTAGATGCCCAATTTG
GAGGCATTGATCAGAGAAAGATTTTCACCTTTGCAGAGAAGTACCTCCCT
GCACTTGGCTATTCAAAACGGGTCCATCTGATGAATCCTATGGTTCCAGG
ATTAACAGGCAGCAAAATGAGCTCTTCAGAAGAGGAGTCCAAGATTGATC
TCCTTGATCGGAAGGAGGATGTGAAGAAAAAACTGAAGAAGGCCTTCTGT
GAGCCAGGAAATGTGGAGAACAATGGGGTTCTGTCCTTCATCAAGCATGT
CCTTTTTCCCCTTAAGTCCGAGTTTGTGATCCTACGAGATGAGAAATGGG
GTGGAAACAAAACCTACACAGCTTACGTGGACCTGGAAAAGGACTTTGCT
GCTGAGGTTGTACATCCTGGAGACCTGAAGAATTCTGTTGAAGTCGCACT
GAACAAGTTGCTGGATCCAATCCGGGAAAAGTTTAATACCCCTGCCCTGA
AAAAACTGGCCAGCGCTGCCTACCCAGATCCCTCAAAGCAGAAGCCAATG
GCCAAAGGCCCTGCCAAGAATTCAGAACCAGAGGAGGTCATCCCATCCCG
GCTGGATATCCGTGTGGGGAAAATCATCACTGTGGAGAAGCACCCAGATG
CAGACAGCCTGTATGTAGAGAAGATTGACGTGGGGGAAGCTGAACCACGG
ACTGTGGTGAGCGGCCTGGTACAGTTCGTGCCCAAGGAGGAACTGCAGGA
CAGGCTGGTAGTGGTGCTGTGCAACCTGAAACCCCAGAAGATGAGAGGAG
TCGAGTCCCAAGGCATGCTTCTGTGTGCTTCTATAGAAGGGATAAACCGC
CAGGTTGAACCTCTGGACCCTCCGGCAGGCTCTGCTCCTGGTGAGCACGT
GTTTGTGAAGGGCTATGAAAAGGGCCAACCAGATGAGGAGCTCAAGCCCA
AGAAGAAAGTCTTCGAGAAGTTGCAGGCTGACTTCAAAATTTCTGAGGAG
TGCATCGCACAGTGGAAGCAAACCAACTTCATGACCAAGCTGGGCTCCAT
TTCCTGTAAATCGCTGAAAGGGGGGAACATTAGCTAG

From GenBank Accession Number NM_{—}003680 (Ribas de Pouplana et al., Proc. Natl. Acad. Sci. USA, 92(1):166-170, 1996).

### 6.10 Neuropilin 1 Polypeptide and Polynucleotide Sequences

(SEQ ID NO:16)

Human Neuropilin 1 Polypeptide
MERGLPLLCAVLALVLAPAGAFRNDKCGDTTKIESPGYLTSPGYPHSYHP
SEKCEWLIQAPDPYQRIMINFNPHFDLEDRDCKYDYVEVFDGENENGHFR
GKFCGKIAPPPVVSSGPFLFIKFVSDYETHGAGFSIRYEIFKRGPECSQN
YTTPSGVIKSPGFPEKYPNSLECTYIVFAPKMSEIILEFESFDLEPDSNP
PGGMFCRYDRLEIWDGFPDVGPHIGRYCGQKTPGRIRSSSGILSMVFYTD
SAIAKEGFSANYSVLQSSVSEDFKCMEALGMESGEIHSDQITASSQYSTN
WSAERSRLNYPENGWTPGEDSYREWIQVDLGLLRFVTAVGTQGAISKETK
KKYYVKTYKIDVSSNGEDWITTKEGNKPVLFQGNTNPTDVVVAVFPKPLI
TRFVRIKPATWETGISMRFEVYGCKITDYPCSGMLGMVSGLISDSQITSS
NQGDRNWMPENIRLVTSRSGWALPPAPHSYINEWLQIDLGEEKIVRGIII
QGGKHRENKVFMRKFKIGYSNNGSDWKMIMDDSKRKAKSFEGNNNYDTPE
LRTFPALSTRFIRIYPERATHGGLGLRMELLGCEVEAPTAGPTTPNGNLV
DECDDDQANCHSGTGDDFQLTGGTTVLATEKPTVIDSTIQSGIK

(SEQ ID NO:33)

DNA Encoding Human Neuropilin 1
ATGGAGAGGGGGCTGCCGCTCCTCTGCGCCGTGCTCGCCCTCGTCCTCGC
CCCGGCCGGCGCTTTTCGCAACGATAAATGTGGCGATACTATAAAAATTG
AAAGCCCCGGGTACCTTACATCTCCTGGTTATCCTCATTCTTATCACCCA
AGTGAAAAATGCGAATGGCTGATTCAGGCTCCGGACCCATACCAGAGAAT
TATGATCAACTTCAACCCTCACTTGATTTGGAGGACAGAGACTGCAAGTA
TGACTACGTGGAAGTCTTCGATGGAGAAAATGAAAATGGACATTTTAGGG
GAAAGTTCTGTGGAAAGATAGCCCCTCCTCCTGTTGTGTCTTCAGGGCCA
TTTCTTTTTATCAAATTTGTCTCTGACTACGAAACACATGGTGCAGGATT
TTCCATACGTTATGAAATTTTCAAGAGAGGTCCTGAATGTTCCCAGAACT
ACACAACACCTAGTGGAGTGATAAAGTCCCCGGATTCCCTGAAAAATATC
CCAACAGCCTTGAATGCACTTATATTGTCTTTGCGCCAAAGATGTCAGAG
ATTATCCTGGAATTTGAAAGCTTTGACCTGGAGCCTGACTCAAATCCTCC
AGGGGGGATGTTCTGTCGCTACGACCGGCTAGAAATCTGGGATGGATTCC
CTGATGTTGGCCCTCACATTGGGCGTTACTGTGGACAGAAAACACCAGGT
CGAATCCGATCCTCATCGGGCATTCTCTCCATGGTTTTTACACCGACAGC
GCGATAGCAAAAGAAGGTTTCTCAGCAAACTACAGTGTCTTGCAGAGCAG
TGTCTCAGAAGATTTCAAATGTATGGAAGCTCTGGGCATGGAATCAGGAG
AAATTCATTCTGACCAGATCACAGCTTCTTCCCAGTATAGCACCAACTGG
TCTGCAGAGCGCTCCCGCCTGAACTACCCTGAGAATGGGTGGACTCCCGG
AGAGGATTCCTACCGAGAGTGGATACAGGTAGACTTGGGCTTCTGCGCTT
TGTCACGGCTGTCGGGACACAGGGCGCCATTTCAAAAGAAACCAAGAAGA
AATATTATGTCAAGACTTACAAGATCGACGTTAGCTCCAACGGGGAAGAC
TGGATCACCATAAAAGAAGGAAACAAACCTGTTCTCTTTCAGGGAAACAC
CAACCCCACAGATGTTGTGGTTGCAGTATTCCCCAAACCACTGATAACTC
GATTTGTCCGAATCAAGCCTGCAACTTGGGAAACTGGCATATCTAGAGAT
TTGAAGTATACGGTTGCAAGATAACAGATTATCCTTGCTCTGGAATGTTG
GGTATGGTGTCTGGACTTATTTCTGACTCCCAGATCACATCATCCAACCA
AGGGGACAGAAACTGGATGCCTGAAAACATCCGCCTGGTAACCAGTCGCT
CTGGCTGGGCACTTCCACCCGCACCTCATTCCTACATCAATGAGTGGCTC
CAAATAGACCTGGGGGAGGAGAAGATCGTGAGGGGCATCATCATTCAGGG
GGGAAGCACCGAGAGAACAAGGTGTTCATGAGGAAGTTCAAGATCGGGTA
CAGCAACAACGGCTCGGACTGGAAGATGATCATGGATGACAGCAAACGCA
AGGCGAAGTCTTTTGAGGGCAACAACAACTATGATACACCTGAGCTGCGG
ACTTTTCCAGCTCTCTCCACGCGATTCATCAGGATCTACCCCGAGAGAGC
CACTCATGGCGGACTGGGGCTCAGAATGGAGCTGCTGGGCTGTGAAGTGG
AAGCCCTACAGCTGGACCGACCACTCCCAACGGGAACTTGGTGGATGAAT
GTGATGACGACCAGGCCAACTGCCACAGTGGAACAGGTGATGACTTCCAG
CTCACAGGTGGCACCACTGTGCTGGCCACAGAAAAGCCCACGGTCATAGA
CAGCACCATACAATCAGGTATCAAATAA

From GenBank Accession Number BC007533.

### 6.11 Interferon-1 Polypeptide and Polynucleotide Sequences

(SEQ ID NO:17)

Human Interferon-Alpha Polypeptide
MASPFALLMVLVVLSCKSSCSLGCDLPETHSLDNRRTLMLLAQMSRISPS
SCLMDRHDFGFPQEEFDGNQFQKAPAISVLHELIQQIFNLFTTKDSSAAW
DEDLLDKFCTELYQQLNDLEACVMQEERVGETPLMNADSILAVKKYFRRI
TLYLTEKKYSPCAWEVVRAEIMRSLSLSTNLQERLRRKE

(SEQ ID NO:34)

DNA Encoding Human Interferon-Alpha
TCATCTGCTGCTTGGGATGAGACCCTCCTAGACAAATTCTACACTGAACT
CTACCAGCAGCTGAATGACCTGGAAGCCTGTGTGATACAGGGGGTGGGGG
TGACAGAGACTCCCCTGATGAAGGAGGACTCCATTCTGGCTGTGAGGAAA
TACTTCCAAAGAATCACTCTCTATCTGAAAGAGAAGAAATACAGCCCTTG
TGCCTGGGAGGTTGTCAGAGCAGAAATCATGAGATCTTTTTCTTTGTCAA
CAAACTTGCAAGAAAGTTTAAGAAGTAAGGAATGAAAACTGGTTCAACAT
GGAAATGATTTTCATTGATTCGTATGCCAGCTCACCTTTTTATGATCTGC
CATTTCAAAGACTCATGTTTCTGCTATGACCATGACACGATTTAAATCTT
TTCAAATGTTTTTACGAGTATTAATCAACATTGTATTCAGCTCTTAAGGC
ACTAGTCCCTTACAGAGGACCATGCTGACTGATCCATTATCTATTTAAAT
ATTTTTAAAATATTATTTATTTAACTATTTATAAAACAACTTATTTTTGT
TCATATTATGTCATGTGCACCTTTGCACAGTGGTTAATGTAATAAAATGT
GTTCTTTGTATTTGGTATATTTATTTTGTGTTGTTCATTGAACTTTTGCT
ATGGAACTTTTGTACTTGTTTATTCTTTAAAATGAAATTCCAAGCCTAAT
TGTGCAACCTGATTACAGAATAACTGGTACACTTCATTTATCCATCAATA
TTATATTCAAGATATAAGTAAAAATAAACTTTCTGTAAACCAGTTG

From GenBank Accession Numbers E00172 and NP_{—}076918.

### 6.12 Kinase Insert Domain Receptor (KDR) Polypeptide and Polynucleotide Sequences

(SEQ ID NO:18)

Human KDR Polypeptide
MQSKVLLAVALWLCVETRAASVGLPSVSLDLPRLSIQKDILTIKANTTLQ
ITCRGQRDLDWLWPNNQSGSEQRVEVTECSDGLFCKTLTIPKVIGNDTGA
YKCFYRETDLASVIYVYVQDYRSPFIASVSDQHGVVYITENKNKTVVIPC
LGSISNLNVSLCARYPEKRFVPDGNRISWDSKKGFTIPSYMISYAGMVFC
EAKINDESYQSIMYIVVVVGYRIYDVVLSPSHGIELSVGEKLVLNCTART
ELNVGIDFNWEYPSSKHQHKKLVNRDLKTQSGSEMKKFLSTLTIDGVTRS
DQGLYTCAASSGLMTKKNSTFVRVHEKPFVAFGSGMESLVEATVGERVRI
PAKYLGYPPPEIKWYKNGIPLESNHTIKAGHVLTIMEVSERDTGNYTVIL
TNPISKEKQSHVVSLVVYVPPQIGEKSLISPVDSYQYGTTQTLTCTVYAI
PPPHHIHWYWQLEEECANEPSQAVSVTNPYPCEEWRSVEDFQGGNKIEVN
KNQFALIEGKNKTVSTLVTQAANVSALYKCEAVNKVGRGERVISFHVTRG
PEITLQPDMQPTEQESVSLWCTADRSTFENLTWYKLGPQPLPIHVGELPT
PVCKNLDTLWKLNATMFSNSTNDILIMELKNASLQDQGDYVCLAQDRKTK
KRHCVVRQLTVLERVAPTITGNLENQTTSIGESIEVSCTASGNPPPQIMW
FKDNETLVEDSGIVLKDGNRNLTIRRVRKEDEGLYTCQACSVLGCAKVEA
FFIIEGAQEKTNLEIIILVGTAVIANFFWLLLVIILRTVKRANGGELKTG
YLSIVMDPDELPLDEHCERLPYDASKWEFPRDRLKLGKPLGRGAFGQVIE
ADAFGIDKTATCRTVAVKMLKEGATHSEHRALMSELKILIHIGHHLNVVN
LLGACTKPGGPLMVIVEFCKFGNLSTYLRSKRNEFVPYKTKGARFRQGKD
YVGATPVDLKRRLDSTTSSQSSASSGFVEEKSLSDVEEEEAPEKLYKDFL
TLEHLICYSFQVAKGMEFLASRKCIHRDLAARNILLSEKNVVKICDFGLA
RDIYKDPDYVRKGDARLPLKWMAPETIFDRVYTIQSDVWSFGVLLWEIFS
LGASPYPGVKIDEEFCRRLKEGTRMRAPDYTTPEMYQTMLDCWHGEPSQR
PTFSELVEHLGNLLQANAQQDGKDYIVLPISETLSMEEDSGLSLPTSPVS
CMEEEEVCDPKFHYDNTAGISQYLQNSKRKSRPVSVKTFEDIPLEEPEVK
VIPDDNQTDSGMVLASEELKTLEDRTKLSPSFGGMVPSKSRESVASEGSN
QTSGYQSGYHSDDTDTTVYSSEEAELLKLIEIGVQTGSTAQILQPDSGTT
LSSPPV

(SEQ ID NO:35)

DNA Encoding Human KDR
ATGCAGAGCAAGGTGCTGCTGGCCGTCGCCCTGTGGCTCTGCGTGGAGAC
CCGGGCCGCCTCTGTGGGTTTGCCTAGTGTTTCTCTTGATCTGCCCAGGC
TCAGCATACAAAAAGACATACTTACAATTAAGGCTAATACAACTCTTCAA
ATTACTTGCAGGGGACAGAGGGACTTGGACTGGCTTTGGCCCAATAATCA
GAGTGGCAGTGAGCAAAGGGTGGAGGTGACTGAGTGCAGCGATGGCCTCT
TCTGTAAGACACTCACAATTCCAAAAGTGATCGGAAATGACACTGGAGCC
TACAAGTGCTTCTACCGGGAAACTGACTTGGCCTCGGTCATTTATGTCTA
TGTTCAAGATTACAGATCTCCATTTATTGCTTCTGTTAGTGACCAACATG
GAGTCGTGTACATTACTGAGAACAAAAACAAAACTGTGGTGATTCCATGT
CTCGGGTCCATTTCAAATCTCAACGTGTCACTTTGTGCAAGATACCCAGA
AAAGAGATTTGTTCCTGATGGTAACAGAATTTCCTGGGACAGCAAGAAGG
GCTTTACTATTCCCAGCTACATGATCAGCTATGCTGGCATGGTCTTCTGT
GAAGCAAAAATTAATGATGAAAGTTACCAGTCTATTATGTACATAGTTGT
CGTTGTAGGGTATAGGATTTATGATGTGGTTCTGAGTCCGTCTCATGGAA
TTGAACTATCTGTTGGAGAAAAGCTTGTCTTAAATTGTACAGCAAGAACT
GAACTAAATGTGGGGATTGACTTCAACTGGGAATACCCTTCTTCGAAGCA
TCAGCATAAGAAACTTGTAAACCGAGACCTAAAAACCCAGTCTGGGAGTG
AGATGAAGAAATTTTTGAGCACCTTAACTATAGATGGTGTAACCCGGAGT
GACCAAGGATTGTACACCTGTGCAGCATCCAGTGGGCTGATGACCAAGAA
GAACAGCACATTTGTCAGGGTCCATGAAAAACCTTTTGTTGCTTTTGGAA
GTGGCATGGAATCTCTGGTGGAAGCCACGGTGGGGGAGCGTGTCAGAATC
CCTGCGAAGTACCTTGGTTACCCACCCCCAGAAATAAAATGGTATAAAAA
TGGAATACCCCTTGAGTCCAATCACACAATTAAAGCGGGGCATGTACTGA
CGATTATGGAAGTGAGTGAAAGAGACACAGGAAATTACACTGTCATCCTT
ACCAATCCCATTTCAAAGGAGAAGCAGAGCCATGTGGTCTCTCTGGTTGT
GTATGTCCCACCCCAGATTGGTGAGAAATCTCTAATCTCTCCTGTGGATT
CCTACCAGTACGGCACCACTCAAACGCTGACATGTACGGTCTATGCCATT
CCTCCCCCGCATCACATCCACTGGTATTGGCAGTTGGAGGAAGAGTGCGC
CAACGAGCCCAGCCAAGCTGTCTCAGTGACAAACCCATACCCTTGTGAAG
AATGGAGAAGTGTGGAGGACTTCCAGGGAGGAAATAAAATTGAAGTTAAT
AAAAATCAATTTGCTCTAATTGAAGGAAAAAACAAAACTGTAAGTACCCT
TGTTATCCAAGCGGCAAATGTGTCAGCTTTGTACAAATGTGAAGCGGTCA
ACAAAGTCGGGAGAGGAGAGAGGGTGATCTCCTTCCACGTGACCAGGGGT
CCTGAAATTACTTTGCAACCTGACATGCAGCCCACTGAGCAGGAGAGCGT
GTCTTTGTGGTGCACTGCAGACAGATCTACGTTTGAGAACCTCACATGGT
ACAAGCTTGGCCCACAGCCTCTGCCAATCCATGTGGGAGAGTTGCCCACA
CCTGTTTGCAAGAACTTGGATACTCTTTGGAAATTGAATGCCACCATGTT
CTCTAATAGCACAAATGACATTTTGATCATGGAGCTTAAGAATGCATCCT
TGCAGGACCAAGGAGACTATGTCTGCCTTGCTCAAGACAGGAAGACCAAG
AAAAGACATTGCGTGGTCAGGCAGCTCACAGTCCTAGAGCGTGTGGCACC
CACGATCACAGGAAACCTGGAGAATCAGACGACAAGTATTGGGGAAAGCA
TCGAAGTCTCATGCACGGCATCTGGGAATCCCCCTCCACAGATCATGTGG
TTTAAAGATAATGAGACCCTTGTAGAAGACTCAGGCATTGTATTGAAGGA
TGGGAACCGGAACCTCACTATCCGCAGAGTGAGGAAGGAGGACGAAGGCC
TCTACACCTGCCAGGCATGCAGTGTTCTTGGCTGTGCAAAAGTGGAGGCA
TTTTTCATAATAGAAGGTGCCCAGGAAAAGACGAACTTGGAAATCATTAT
TCTAGTAGGCACGGCGGTGATTGCCATGTTCTTCTGGCTACTTCTTGTCA
TCATCCTACGGACCGTTAAGCGGGCCAATGGAGGGGAACTGAAGACAGGC
TACTTGTCCATCGTCATGGATCCAGATGAACTCCCATTGGATGAACATTG
TGAACGACTGCCTTATGATGCCAGCAAATGGGAATTCCCCAGAGACCGGC
TGAAGCTAGGTAAGCCTCTTGGCCGTGGTGCCTTTGGCCAAGTGATTGAA
GCAGATGCCTTTGGAATTGACAAGACAGCAACTTGCAGGACAGTAGCAGT
CAAAATGTTGAAAGAAGGAGCAACACACAGTGAGCATCGAGCTCTCATGT
CTGAACTCAAGATCCTCATTCATATTGGTCACCATCTCAATGTGGTCAAC
CTTCTAGGTGCCTGTACCAAGCCAGGAGGGCCACTCATGGTGATTGTGGA
ATTCTGCAAATTTGGAAACCTGTCCACTTACCTGAGGAGCAAGAGAAATG
AATTTGTCCCCTACAAGACCAAAGGGGCACGATTCCGTCAAGGGAAAGAC
TACGTTGGAGCAATCCCTGTGGATCTGAAACGGCGCTTGGACAGCATCAC
CAGTAGCCAGAGCTCAGCCAGCTCTGGATTTGTGGAGGAGAAGTCCCTCA
GTGATGTAGAAGAAGAGGAAGCTCCTGAAGATCTGTATAAGGACTTCCTG
ACCTTGGAGCATCTCATCTGTTACAGCTTCCAAGTGGCTAAGGGCATGGA
GTTCTTGGCATCGCGAAAGTGTATCCACAGGGACCTGGCGGCACGAAATA
TCCTCTTATCGGAGAAGAACGTGGTTAAAATCTGTGACTTTGGCTTGGCC
CGGGATATTTATAAAGATCCAGATTATGTCAGAAAAGGAGATGCTCGCCT
CCCTTTGAAATGGATGGCCCCAGAAACAATTTTTGACAGAGTGTACACAA
TCCAGAGTGACGTCTGGTCTTTTGGTGTTTTGCTGTGGGAAATATTTTCC
TTAGGTGCTTCTCCATATCCTGGGGTAAAGATTGATGAAGAATTTTGTAG
GCGATTGAAAGAAGGAACTAGAATGAGGGCCCCTGATTATACTACACCAG
AAATGTACCAGACCATGCTGGACTGCTGGCACGGGGAGCCCAGTCAGAGA
CCCACGTTTTCAGAGTTGGTGGAACATTTGGGAAATCTCTTGCAAGCTAA
TGCTCAGCAGGATGGCAAAGACTACATTGTTCTTCCGATATCAGAGACTT
TGAGCATGGAAGAGGATTCTGGACTCTCTCTGCCTACCTCACCTGTTTCC
TGTATGGAGGAGGAGGAAGTATGTGACCCCAAATTCCATTATGACAACAC
AGCAGGAATCAGTCAGTATCTGCAGAACAGTAAGCGAAAGAGCCGGCCTG
TGAGTGTAAAAACATTTGAAGATATCCCGTTAGAAGAACCAGAAGTAAAA
GTAATCCCAGATGACAACCAGACGGACAGTGGTATGGTTCTTGCCTCAGA
AGAGCTGAAAACTTTGGAAGACAGAACCAAATTATCTCCATCTTTTGGTG
GAATGGTGCCCAGCAAAAGCAGGGAGTCTGTGGCATCTGAAGGCTCAAAC
CAGACAAGCGGCTACCAGTCCGGATATCACTCCGATGACACAGACACCAC
CGTGTACTCCAGTGAGGAAGCAGAACTTTTAAAGCTGATAGAGATTGGAG
TGCAAACCGGTAGCACAGCCCAGATTCTCCAGCCTGACTCGGGGACCACA
CTGAGCTCTCCTCCTGTTTAA

From GenBank Accession Number NM_{—}002253 Terman et al., Oncogene, 6(9):1677-1683, 1991).

### 7. REFERENCES

The following references, to the extent that they provide exemplary procedural or other details supplementary to those set forth herein, are specifically incorporated herein by reference in whole or in part:
Araki, Taniwaki, Becerra, Chader and Schwartz, “Pigment epithelium-derived factor (PEDF) differentially protects immature but not mature cerebellar granule cells against apoptotic cell death,” J. Neurosci. Res., 53:7-15, 1998.
Bennett, Maguire, Cideciyan et al., “Stable transgene expression in rod photoreceptors after recombinant adeno-associated virus-mediated gene transfer to monkey retina,” Proc. Natl. Acad. Sci. USA, 96:9920-25, 1999.
Bilak, Corse, Bilak, Lehar, Tombran-Tink and Kuncl, “Pigment epithelium-derived factor (PEDF) protects motor neurons from chronic glutamate-mediated neurodegeneration,” J. Neuropathol. Exp. Neurol., 58:719-28, 1999.
Cao, Tombrin-Tink, Chen, Mrazek, Elias and McGinnis, “Pigment epithelium-derived factor protects cultured retinal neurons against hydrogen peroxide-induced cell death,” J. Neurosci. Res., 57:789-800, 1999.
Dawson, Volpert, Gillis et al., “Pigment epithelium-derived factor: a potent inhibitor of angiogenesis,” Science, 285:245-48, 1999.
DeCoster, Schabelman, Tombran-Tink and Bazan, “Neuroprotection by pigment epithelial-derived factor against glutamate toxicity in developing primary hippocampal neurons,” J. Neurosci. Res., 56:604-10, 1999.
Edelman and Castro, “Visualization and quantitative digital image analysis of experimental choroidal neovascularization (CNV) using FITC-dextran labeled flat mounts,” Invest. Ophthalmol. Vis. Sci., 41 :S834, 2000.
Good, Polverini, Rastinejad et al., “A tumor suppressor-dependent inhibitor of angiogenesis is immunologically and functionally indistinguishable from a fragment of thrombospondin,” Proc. Natl. Acad. Sci. USA, 87:6624-28, 1990.
Guyer, Martin, Klein, Haller and Group, “Anti-VEGF therapy in patients with exudative age-related macular degeneration,” Invest. Ophthalmol. Vis. Sci., 42:S522, 2001.
Houenou, D'Costa, Li et al., “Pigment epithelium derived factor promotes the survival and differentiation of developing spinal motor neurons,” J. Comp. Neurol., 412:506-14, 1999.
Jablonski, Tombran-Tink, Mrazek and Iannoaccone, “Pigment epithelium-derived factor supports normal development of photoreceptor neurons and opsin expression after retinal pigment epithelium removal,” J. Neurosci., 20:7149-57, 2000.
Kwak, Okamoto, Wood and Campochiaro, “VEGF is an important stimulator in a model of choroidal neovascularization,” Invest. Ophthalmol. Vis. Sci., 41:3158-64, 2000.
Lau, McGee, Zhou et al., “Retinal degeneration is slowed in transgenic rats by AAV-mediated delivery of FGF-2,” Invest. Ophthalmol. Vis. Sci., 41:3622-33, 2000.
The Macular Photocoagulation Study Group, “Argon laser photocoagulation for neovascular maculopathy: Five year results from randomized clinical trials,” Arch Ophthalmol., 109:1109-14, 1991.
Maione, Gray, Petro et al., “Inhibition of angiogenesis by recombinant human platelet factor-4 and related peptides,” Science, 247:77-79, 1990.
Mori, Duh, Gehlbach et al., “Pigment epithelium-derived factor inhibits retinal and choroidal neovascularization,” J. Cell Physiol., 188:253-63, 2001a.
Mori, Ando, Gehlbach et al., “Inhibition of choroidal neovascularization by intravenous injection of adenoviral vectors expressing secretable endostatin,” Amer. J. Pathol., 159:313-20, 2001b.
O'Reilly, Holmgren, Shing et al., “Angiostatin: a novel angiogenesis inhibitor that mediates the suppression of metastases by a Lewis lung carcinoma,” Cell, 79:315-28, 1994.
O'Reilly, Boehm, Shing et al., “Endostatin: an endogenous inhibitor of angiogenesis and tumor growth,” Cell, 88:277-85, 1997.
O'Reilly, Pirie-Sheherd, Lane and Folkman, “Antiangiogenic activity of the cleaved conformation of the serpin antithrombin,” Science, 285:1926-28, 1999.
Ozaki, Seo, Ozaki et al., “Blockade of vascular endothelial cell growth factor receptor signaling is sufficient to completely prevent retinal neovascularization,” Am. J. Pathol., 156:679-707, 2000.
Schwartz, Blumenkranz, Rosenfeld et al., “Safety of rhuFab V2, an anti-VEGF antibody fragment, as a single intravitreal injection in subjects with neovascular age-related macular degeneration,” Invest. Ophthalmol. Vis. Sci., 42:S522, 2001.
Seo, Kwak, Ozaki et al., “Dramatic inhibition of retinal and choroidal neovascularization by oral administration of a kinase inhibitor,” Amer. J. Pathol., 154:1743-53, 1999.
Steele, Chader, Johnson and Tombran-Tink, “Pigment epithelium-derived factor: neurotrophic activity and identification as a member or the serine protease inhibitor gene family,” Proc. Natl. Acad. Sci. USA, 90:1526-30, 1993.
Stellmach, Crawford, Zhou and Bouck, “Prevention of ischemia-induced retinopathy by the natural ocular antiangiogenic agent pigment epithelium-derived factor,” Proc. Natl. Acad. Sci. USA, 98:2593-97, 2001.
Taniwaki, Becerra, Chader and Schwartz, “Pigment epithelium-derived factor is a survival factor for cerebellar granule cells in culture,” J. Neurochem., 64:2509-17, 1995.
Tobe, Ortega, Luna et al., “Targeted disruption of the FGF2 gene does not prevent choroidal neovascularization in a murine model,” Am. J. Pathol., 153:1641-46, 1998a.
Tobe, Okamoto, Vinores et al., “Evolution of neovascularization in mice with overexpression of vascular endothelial growth factor in photoreceptors,” Invest. Ophthalmol. Vis. Sci., 39:180-88, 1998b.
Tombran-Tink, Chader and Johnson, “PEDF: a pigment epithelium derived factor with potent neuronal differentiating activity,” Exp. Eye Res., 53:411-14, 1991.
Zolotukhin, Potter, Hauswirth, Guy and Muzyczka, “A ‘humanized’ green fluorescent protein cDNA adapted for high-level expression in mammalian cells,” J. Virol., 70:4646-54, 1996.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. An adeno-associated viral (AAV) vector comprising a polynucleotide that comprises a nucleic acid segment that encodes a choroidal or ocular neovascularization inhibitory polypeptide operably linked to a promoter that expresses said segment to produce said polypeptide in a selected mammalian host cell.

2.-46. (canceled)

47. The adeno-associated viral vector of claim 1, wherein said choroidal or said ocular neovascularization inhibitory polypeptide is pigment epithelium-derived factor (PEDF) or angiostatin.

48. The adeno-associated viral vector of claim 47, wherein said choroidal or said ocular neovascularization inhibitory polypeptide is PEDF.

49. The adeno-associated viral vector of claim 47, wherein said choroidal or said ocular neovascularization inhibitory polypeptide is angiostatin.

50. The adeno-associated viral vector of claim 1, wherein said promoter is a β-actin promoter.

51. The adeno-associated viral vector of claim 1, wherein said polynucleotide further comprises a 5′ regulatory element operably linked to said nucleic acid segment.

52. The adeno-associated viral vector of claim 51, wherein said 5′ regulatory element comprises a CMV enhancer.

53. The adeno-associated viral vector of claim 1, wherein said polynucleotide further comprises a 3′ regulatory element operably linked to said nucleic acid segment.

54. The adeno-associated viral vector of claim 53, wherein said 3′ regulatory element comprises a woodchuck hepatitis virus post-transcriptional regulatory element (WPRE).

55. The adeno-associated viral vector of claim 47, wherein said nucleic acid segment encodes a polypeptide that is at least 98% identical to the sequence of SEQ ID NO:1 or SEQ ID NO:4, and that has neovascularization inhibitory activity when administered to a mammalian eye.

56. The adeno-associated viral vector of claim 55, wherein said nucleic acid segment encodes a polypeptide that comprises the sequence of SEQ ID NO: 1 or SEQ ID NO:4.

57. A virion or viral particle comprising the adeno-associated viral vector of claim 1.

58. The virion or viral particle of claim 57, comprised within a pharmaceutical vehicle.

59. A mammalian host cell comprising the adeno-associated viral vector of claim 1 or the virion or viral particle of claim 57.

60. The mammalian host cell of claim 59, wherein said host cell is an eye cell, a scleral cell, a choroidal cell, or a retinal cell.

61. The mammalian host cell of claim 60, wherein said host cell is a human host cell.

62. A composition comprising:
(a) the adeno-associated viral vector of claim 1;
(b) the virion or viral particle of claim 57; or (c) the mammalian host cell of claim 59.

63. A method of providing a therapeutically effective amount of a choroidal or ocular neovascularization inhibitory polypeptide to a mammal in need thereof, said method comprising the step of providing to said mammal the composition of claim 62, in an amount and for a time effective to provide said therapeutically effective amount of said choroidal or said ocular neovascularization-inhibitory polypeptide to said mammal.

64. The method of claim 63, wherein said composition is provided to said mammal systemically, or by direct or indirect administration to a cell, tissue, or organ of said mammal.

65. The method of claim 63, wherein said composition is provided to mammal by ocular injection, intravitreolar injection, retinal injection, or subretinal injection.

66. A method of treating choroidal or ocular neovascularization in a mammal, said method comprising the step of providing to a mammal in need thereof the composition of claim 62, in an amount and for a time effective to treat said choroidal or said ocular neovascularization in said mammal.

67. The method of claim 66, wherein said composition is provided to said mammal systemically, or by direct or indirect administration to a cell, tissue, or organ of said mammal.

68. The method of claim 66, wherein said composition is provided to mammal by ocular injection, intravitreolar injection, retinal injection, or subretinal injection.
